(19) United States
(12) Patent Application Publication
(10) Pub. No.: US 2026/0360089 A1
(43) Pub. Date: Nov. 12, 2026

(54) Systems and Methods for Extended Reality Visualization of Medical Instrument Placement

(71) Applicant: Bard Access Systems, Inc., Salt Lake City, UT (US)

(72) Inventor: Tyler L. Durfee, Stansbury, UT (US)

(21) Appl. No.: 19/202,972

(22) Filed: May 8, 2025

(51) Int. Cl. G06T 19/00; A61B 90/00; G16H 40/63

(52) U.S. Cl. CPC G06T 19/006; A61B 90/36; G16H 40/63; A61B 2090/365

(57) ABSTRACT

Disclosed herein is a system, method, and apparatus directed to display of extended reality (XR) views including a virtual representation of a medical instrument. One implementation includes an XR apparatus that includes a housing coupled to a display, a head strap, one or more processors, and storage containing logic configured to causes operations including generating a three-dimensional (3D) mesh of a medical procedure environment by performing environmental scanning, identifying an artifact within the medical procedure environment, establishing an anchor position of the artifact within the 3D mesh, continuously receiving reflected signal data corresponding to positioning of the medical instrument, and generating the virtual representation of the medical instrument in the medical procedure environment on the display of the XR headset including: mapping the reflected signal data to the anchor position of the artifact, and mapping changes in strain of the reflected signal data to coordinates within the 3D mesh.

### BACKGROUND

When placing a medical instrument in the peripheral vasculature such as the vasculature of the arms or legs, it is difficult to determine where the medical instrument is at any given point in time. For example, clinicians often use fluoroscopy to track medical instruments such as guidewires or catheters, but the vasculature is not visible in such X-ray-based technology, which makes it is impossible to determine exactly where the tip of a guidewire or catheter is in the vasculature. In addition, fluoroscopy exposes both patients and clinicians to ionizing radiation putting their health at risk. Therefore, an ability to visualize anatomy such as the peripheral vasculature is needed. In addition, an ability to visualize such anatomy in conjunction with medical instruments such as guidewires and catheters is needed to finally make it possible to determine exactly where such medical instruments are during placement thereof. Lastly, such abilities should not adversely affect patients or clinicians.

Disclosed herein are systems and methods for locating medical instruments or placing medical instruments that address one or more needs such as the foregoing.

In the past, certain intravascular guidance of medical instruments, such as guidewires and catheters for example, have used fluoroscopic methods for tracking tips of the medical instruments and determining whether distal tips are appropriately localized in their target anatomical structures. However, such fluoroscopic methods expose patients and their attending clinicians to harmful X-ray radiation. Moreover, in some cases, the patients are exposed to potentially harmful contrast media needed for the fluoroscopic methods.

### SUMMARY

Briefly summarized, embodiments disclosed herein are directed to systems, apparatus, computerized methods, and general methods for providing tracking information of a medical instrument using optical fiber technology and generating an extended reality display illustrating the propagation of the medical instrument through a patient vasculature. The systems, apparatus, and method discussed herein may include an introducer configured to provide controlled access to a blood vessel by creating a stable entry point into the vessel through which the medical instrument passes. Specific embodiments include an introducer having specific markings thereon that are identifiable by an extended reality (XR) visualization device, which allow the XR visualization device or accompanying XR visualization logic to create an anchor position within the medical environment. The anchor position may then be utilized to facilitate shape sensing of the medical instrument as it propagates through the patient vasculature using an optical fiber within the medical instrument.

In some embodiments, the medical instrument includes the optical fiber having one or more optical fiber cores, where each are configured with an array of sensors (reflective gratings), which are spatially distributed over a prescribed length of the core fiber to generally sense external strain and temperature on those regions of the core fiber occupied by the sensor. Each optical fiber core is configured to receive light (e.g., broadband) from a console during advancement through the vasculature of a patient, where the broadband light propagates along at least a partial distance of the optical fiber core toward the distal end. Given that each sensor positioned along the optical fiber core is configured to reflect light of a different, specific spectral width, the array of sensors enables distributed measurements throughout the prescribed length of the medical instrument. These distributed measurements may include wavelength shifts having a correlation with strain and/or temperature experienced by the sensor.

The reflected light from the sensors (reflective gratings) within an optical fiber core is returned from the medical instrument for processing by the console. The physical state of the medical instrument may be ascertained based on analytics of the wavelength shifts of the reflected light. For example, the strain caused through bending of the medical instrument and hence angular modification of the optical fiber core, causes different degrees of deformation. The different degrees of deformation alter the shape of the sensors (reflective grating) positioned on the optical fiber core, which may cause variations (shifts) in the wavelength of the reflected light from the sensors positioned on the optical fiber core. The optical fiber core may comprise a single optical fiber, or a plurality of optical fibers (in which case, the optical fiber core is referred to as a “multi-core optical fiber”).

As used herein, the term “core fiber,” generally refers to a single optical fiber core disposed within a medical instrument. Thus, discussion of a core fiber refers to single optical fiber core and discussion of a multi-core optical fiber refers to a plurality of core fibers. Various embodiments discussed below to detection of the health (and particularly the damage) that occurs in each of an optical fiber core of medical instrument including (i) a single core fiber, and (ii) a plurality of core fibers. It is noted that in addition to strain altering the shape of a sensor, ambient temperature variations may also alter the shape of a sensor, thereby causing variations (shifts) in the wavelength of the reflected light from the sensors positioned on the optical fiber core.

Specific embodiments of the disclosure include utilization of a medical instrument, such as a stylet, featuring a multi-core optical fiber and a conductive medium that collectively operate for tracking placement with a body of a patient of the stylet or another medical instrument (such as a catheter) in which the stylet is disposed. In lieu of a stylet, a guidewire may be utilized. For convenience, embodiments are generally discussed where the optical fiber core is disposed within a stylet; however, the disclosure is not intended to be so limited as the functionality involving detection of the health of an optical fiber core disclosed herein may be implemented regardless of the medical instrument in which the optical fiber core is disposed. In some embodiments, the optical fiber core may be integrated directly into a wall of the catheter.

In some embodiments, the optical fiber core of a stylet is configured to return information for use in identifying its physical state (e.g., shape length, shape, and/or form) of (i) a portion of the stylet (e.g., tip, segment of stylet, etc.) or a portion of a catheter inclusive of at least a portion of the stylet (e.g., tip, segment of catheter, etc.) or (ii) the entirety or a substantial portion of the stylet or catheter within the body of a patient (hereinafter, described as the “physical state of the stylet” or the “physical state of the catheter”). According to one embodiment of the disclosure, the returned information may be obtained from reflected light signals of different spectral widths, where each reflected light signal corresponds to a portion of broadband incident light propagating along a core of the multi-core optical fiber (core fiber) that is reflected back over the core fiber by a particular sensor located on the core fiber. One illustrative example of the returned information may pertain to a change in signal characteristics of the reflected light signal returned from the sensor, where wavelength shift is correlated to (mechanical) strain on the core fiber or a detected change in ambient temperature.

In some embodiments, the core fiber utilizes a plurality of sensors and each sensor is configured to reflect a different spectral range of the incident light (e.g., different light frequency range). Based on the type and degree of strain asserted on each core fiber, the sensors associated with that core fiber may alter (shift) the wavelength of the reflected light to convey the type and degree of stain on that core fiber at those locations of the stylet occupied by the sensors. The sensors are spatially distributed at various locations of the core fiber between a proximal end and a distal end of the stylet so that shape sensing of the stylet may be conducted based on analytics of the wavelength shifts. Herein, the shape sensing functionality is paired with the ability to simultaneously pass an electrical signal through the same member (stylet) through conductive medium included as part of the stylet.

Similarly, the sensors may alter (shift) the wavelength of the reflected light to convey sensed variations in ambient temperature. The alterations in response to detected variations in ambient temperature thereby provide for a temperature sensing functionality.

More specifically, in some embodiments each core fiber of the multi-core optical fiber is configured with an array of sensors, which are spatially distributed over a prescribed length of the core fiber to generally sense external strain on or variations in ambient temperature proximate those regions of the core fiber occupied by the sensor. Given that each sensor positioned along the same core fiber is configured to reflect light of a different, specific spectral width, the array of sensors enables distributed measurements throughout the prescribed length of the multi-core optical fiber. These distributed measurements may include wavelength shifts having a correlation with strain experienced and/or temperature variations detected by the sensor.

In more detail, each sensor may operate as a reflective grating such as a fiber Bragg grating (FBG), namely an intrinsic sensor corresponding to a permanent, periodic refractive index change inscribed into the core fiber. Stated differently, the sensor operates as a light reflective mirror for a specific spectral width (e.g., a specific wavelength or specific range of wavelengths). As a result, as broadband incident light is supplied by an optical light source and propagates through a particular core fiber, upon reaching a first sensor of the distributed array of sensors for that core fiber, light of a prescribed spectral width associated with the first sensor is reflected back to an optical receiver within a console, including a display and the optical light source. The remaining spectrum of the incident light continues propagation through the core fiber toward a distal end of the stylet. The remaining spectrum of the incident light may encounter other sensors from the distributed array of sensors, where each of these sensors is fabricated to reflect light with different specific spectral widths to provide distributed measurements, as described above.

During operation, multiple light reflections (also referred to as “reflected light signals”) are returned to the console from each of the plurality of core fibers of the multi-core optical fiber. Each reflected light signal may be uniquely associated with a different spectral width. Information associated with the reflected light signals may be used to determine a three-dimensional representation of the physical state of the stylet within the body of a patient through detection of strain in response to emitted incident light. Herein, the core fibers are spatially separated with the cladding of the multi-mode optical fiber and each core fiber is configured to separately return light of different spectral widths (e.g., specific light wavelength or a range of light wavelengths) reflected from the distributed array of sensors fabricated in each of the core fibers.

During vasculature insertion and advancement of the catheter, the clinician may rely on the console to visualize a current physical state (e.g., shape) of a catheter guided by the stylet to avoid potential path deviations. As the periphery core fibers reside at spatially different locations within the cladding of the multi-mode optical fiber, changes in angular orientation (such as bending with respect to the center core fiber, etc.) of the stylet imposes different types (e.g., compression or tension) and degrees of strain on each of the periphery core fibers as well as the center core fiber. The different types and/or degree of strain may cause the sensors of the core fibers to apply different wavelength shifts, which can be measured to extrapolate the physical state of the stylet (catheter).

Additionally, in some embodiments, a medical instrument to be inserted is an implantable medical instrument, such as an implantable catheter that is configured to couple with a port. A second medical instrument that is optically-enabled may be utilized to provide a clinician with a graphical display of the advancement of the implantable medical instrument during the insertion operation. For instance, the second medical instrument may be an optically-enabled stylet that is inserted within the implantable catheter for the insertion operation. Thus, as the insertion operation progresses, incident light signals are provided to the optically-enabled stylet and reflected light signals are returned to a console for analysis. The analysis of the reflected light signals may enable a determination of a shape, positioning, orientation, etc., of the stylet, and consequently, of the implantable catheter. A graphic may then be rendered illustrating the progress of the advancement of the stylet and/or the implantable catheter, optionally as an overlay on a graphic of a patient body. Thus, the clinician may visualize the advancement of the implantable catheter.

Further, as positioning of a properly implanted catheter is generally known, a console may have stored thereon, or access to, desired placements of an implantable catheter (e.g., the desired shape and positioning) that may be utilized in analyses performed by logic of the console to determine a progress and/or an accuracy of the insertion operation as compared to the selected desired placement. In some embodiments, machine-learning techniques may be utilized to determine the progress and/or accuracy.

Herein, some embodiments disclose a computerized method for displaying a virtual representation of a medical instrument on a display of an extended reality (XR) headset, the computerized method comprising generating a three-dimensional (3D) mesh of a medical procedure environment by performing environmental scanning, identifying an artifact within the medical procedure environment, establishing an anchor position of the artifact within the 3D mesh, continuously receiving reflected signal data corresponding to positioning of the medical instrument, and generating the virtual representation of the medical instrument in the medical procedure environment on the display of the XR headset including mapping the reflected signal data to the anchor position of the artifact, and mapping changes in strain of the reflected signal data to coordinates within the 3D mesh.

In some embodiments, the artifact is an introducer disposed adjacent to an insertion site for a vascular access procedure. In some instances, the artifact includes markings, and wherein identifying the artifact includes detecting the markings using computer vision techniques. In some examples, generating the 3D mesh includes performing the environmental scanning by capturing image data with one or more cameras of the XR headset.

In some embodiment, the medical instrument includes a multi-core optical fiber including a plurality of core fibers that generate the reflected signal data. Each of the plurality of core fibers may include a plurality of sensors distributed along a longitudinal length of a corresponding core fiber and each sensor of the plurality of sensors being configured to (i) reflect a light signal of a different spectral width based on received incident light, and (ii) change a characteristic of the reflected light signal based on strain experienced by the core fiber. Further, the virtual representation of the medical instrument may represent shape and placement of a portion of the medical instrument that is inside a vasculature of a patient during a vascular access procedure.

Some embodiments disclose an extended reality (XR) apparatus comprising a housing coupled to a display, a head strap, and one or more cameras, one or more processors housed within the housing, and non-transitory computer-readable medium having stored thereon logic that, when executed by one or more processors, causes operations for displaying a virtual representation of a medical instrument on the display, the operations including generating a three-dimensional (3D) mesh of a medical procedure environment by performing environmental scanning, identifying an artifact within the medical procedure environment, establishing an anchor position of the artifact within the 3D mesh, continuously receiving reflected signal data corresponding to positioning of the medical instrument, and generating the virtual representation of the medical instrument in the medical procedure environment on the display of the XR headset including mapping the reflected signal data to the anchor position of the artifact, and mapping changes in strain of the reflected signal data to coordinates within the 3D mesh.

Yet other embodiments disclose a non-transitory computer-readable medium having stored thereon logic that, when executed by one or more processors, causes operations for displaying a virtual representation of a medical instrument on a display of an extended reality (XR) headset, the operations including generating a three-dimensional (3D) mesh of a medical procedure environment by performing environmental scanning, identifying an artifact within the medical procedure environment, establishing an anchor position of the artifact within the 3D mesh, continuously receiving reflected signal data corresponding to positioning of the medical instrument, and generating the virtual representation of the medical instrument in the medical procedure environment on the display of the XR headset including mapping the reflected signal data to the anchor position of the artifact, and mapping changes in strain of the reflected signal data to coordinates within the 3D mesh.

These and other features of the concepts provided herein will become more apparent to those of skill in the art in view of the accompanying drawings and following description, which disclose particular embodiments of such concepts in greater detail.

### BRIEF DESCRIPTION OF THE DRAWINGS

Embodiments of the disclosure are illustrated by way of example and not by way of limitation in the figures of the accompanying drawings, in which like references indicate similar elements and in which:

FIG. 1A is an illustrative embodiment of a medical instrument monitoring system including a medical instrument with optic shape sensing and fiber optic-based oximetry capabilities in accordance with some embodiments;

FIG. 1B is an alternative illustrative embodiment of the medical instrument monitoring system 100 in accordance with some embodiments;

FIG. 2 is an exemplary embodiment of a structure of a section of the multi-core optical fiber included within the stylet 120 of FIG. 1A in accordance with some embodiments;

FIG. 3A is a first exemplary embodiment of the stylet of FIG. 1A supporting both an optical and electrical signaling in accordance with some embodiments;

FIG. 3B is a cross sectional view of the stylet of FIG. 3A in accordance with some embodiments;

FIG. 4A is a second exemplary embodiment of the stylet of FIG. 1B in accordance with some embodiments;

FIG. 4B is a cross sectional view of the stylet of FIG. 4A in accordance with some embodiments;

FIG. 5A is an elevation view of a first illustrative embodiment of a catheter including integrated tubing, a diametrically disposed septum, and micro-lumens formed within the tubing and septum in accordance with some embodiments;

FIG. 5B is a perspective view of the first illustrative embodiment of the catheter of FIG. 5A including core fibers installed within the micro-lumens in accordance with some embodiments;

FIGS. 6A-6B are flowcharts of the methods of operations conducted by the medical instrument monitoring system of FIGS. 1A-1B to achieve optic 3D shape sensing in accordance with some embodiments;

FIG. 7 is an exemplary embodiment of the medical instrument monitoring system of FIGS. 1A-1B during operation and insertion of the catheter into a patient in accordance with some embodiments;

FIG. 8A is an example introducer including a set of markings disposed thereon in accordance with some embodiments;

FIG. 8B illustrates the example introducer of FIG. 8A coupled with a needle having a medical instrument threaded therethrough in accordance with some embodiments;

FIG. 9A is an illustrative example of an extended reality headset in accordance with some embodiments;

FIG. 9B is a flowchart of a set of operations conducted by the XR headset of FIG. 9A in identifying an introducer and determining an anchor position within a medical procedure environment in accordance with some embodiments;

FIG. 10A is an illustration of an introducer guiding a needle into a patient vasculature and a medical instrument threaded through the needle in accordance with some embodiments;

FIG. 10B is a second illustration of the introducer of FIG. 10A guiding the needle into the patient vasculature and the medical instrument threaded through the needle in accordance with some embodiments;

FIG. 10C is a third illustration of the introducer of FIG. 10A guiding the needle into the patient vasculature and the medical instrument threaded through the needle including an virtual representation of portions of the needle and the medical instrument in accordance with some embodiments;

FIG. 10D is a fourth illustration of the introducer of FIG. 10A guiding the needle into the patient vasculature and the medical instrument threaded through the needle including a second virtual representation of portions of the needle and the medical instrument in accordance with some embodiments;

FIG. 10E is a fifth illustration of the introducer of FIG. 10A guiding the needle into the patient vasculature and the medical instrument threaded through the needle including a virtual representation of portions of the needle, the medical instrument, and the patient vasculature in accordance with some embodiments;

FIG. 11A illustrates a medical environment including a patient under a drape undergoing a vascular access procedure in accordance with some embodiments;

FIG. 11B illustrates a view of the medical environment of FIG. 11A through an XR headset that includes a virtual representation of the medical instrument propagating through the patient vasculature in accordance with some embodiments;

FIG. 11C is a detailed view of a portion of FIG. 11B in accordance with some embodiments;

FIG. 12 is a conceptual virtual reality (VR) view of a patient vasculature and a virtual representation of a medical instrument propagating through the patient vasculature during a vascular access procedure in accordance with some embodiments;

FIG. 13 illustrates a view of a medical environment of FIG. 11A through an XR headset that includes a virtual reality (VR) view of a patient vasculature and a virtual representation of a medical instrument propagating through the patient vasculature during a vascular access procedure overlaid on the patient undergoing the vascular access procedure in accordance with some embodiments; and

FIG. 14 is a flowchart illustrating operations performed in identifying an introducer within a 3D space as an anchor position and generating an EX display of a medical instrument originating from the anchor position in accordance with some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a probe disclosed herein includes a portion of the probe intended to be near a clinician when the probe is used on a patient. Likewise, a “proximal length” of, for example, the probe includes a length of the probe intended to be near the clinician when the probe is used on the patient. A “proximal end” of, for example, the probe includes an end of the probe intended to be near the clinician when the probe is used on the patient. The proximal portion, the proximal end portion, or the proximal length of the probe can include the proximal end of the probe; however, the proximal portion, the proximal end portion, or the proximal length of the probe need not include the proximal end of the probe. That is, unless context suggests otherwise, the proximal portion, the proximal end portion, or the proximal length of the probe is not a terminal portion or terminal length of the probe.

With respect to “distal,” a “distal portion” or a “distal end portion” of, for example, a probe disclosed herein includes a portion of the probe intended to be near or in a patient when the probe is used on the patient. Likewise, a “distal length” of, for example, the probe includes a length of the probe intended to be near or in the patient when the probe is used on the patient. A “distal end” of, for example, the probe includes an end of the probe intended to be near or in the patient when the probe is used on the patient. The distal portion, the distal end portion, or the distal length of the probe can include the distal end of the probe; however, the distal portion, the distal end portion, or the distal length of the probe need not include the distal end of the probe. That is, unless context suggests otherwise, the distal portion, the distal end portion, or the distal length of the probe is not a terminal portion or terminal length of the probe.

The term “logic” may be representative of hardware, firmware or software that is configured to perform one or more functions. As hardware, the term logic may refer to or include circuitry having data processing and/or storage functionality. Examples of such circuitry may include, but are not limited or restricted to, a hardware processor (e.g., microprocessor, one or more processor cores, a digital signal processor, a programmable gate array, a microcontroller, an application specific integrated circuit “ASIC”, etc.), a semiconductor memory, or combinatorial elements.

Additionally, or in the alternative, the term logic may refer to or include software such as one or more processes, one or more instances, Application Programming Interface(s) (API), subroutine(s), function(s), applet(s), servlet(s), routine(s), source code, object code, shared library/dynamic link library (dll), or even one or more instructions. This software may be stored in any type of a suitable non-transitory storage medium, or transitory storage medium (e.g., electrical, optical, acoustical or other form of propagated signals such as carrier waves, infrared signals, or digital signals). Examples of a non-transitory storage medium may include, but are not limited or restricted to a programmable circuit; non-persistent storage such as volatile memory (e.g., any type of random access memory “RAM”); or persistent storage such as non-volatile memory (e.g., read-only memory “ROM”, power-backed RAM, flash memory, phase-change memory, etc.), a solid-state drive, hard disk drive, an optical disc drive, or a portable memory device. As firmware, the logic may be stored in persistent storage.

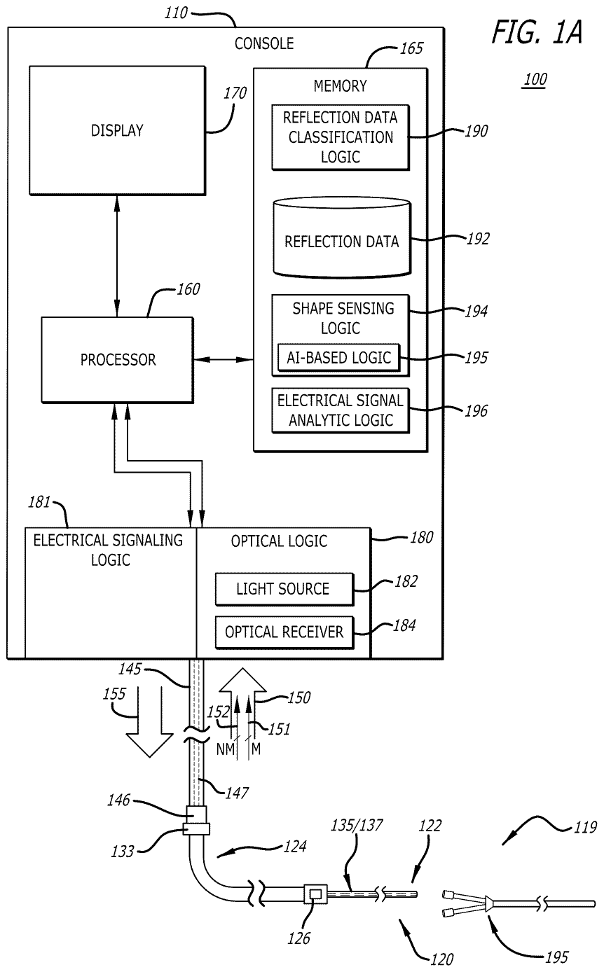

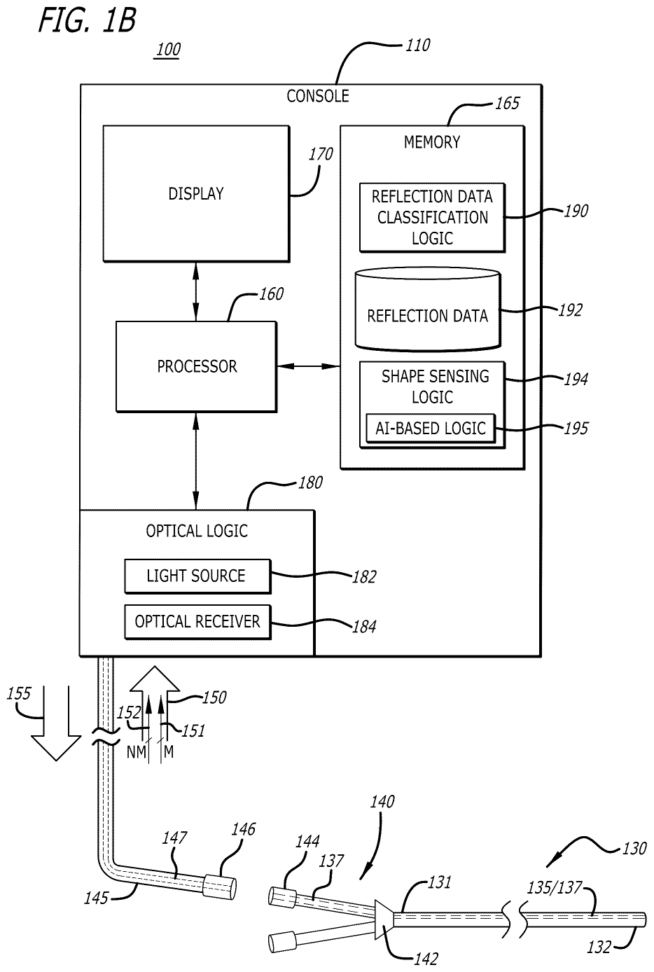

Referring to FIG. 1A, an illustrative embodiment of a medical instrument monitoring system including a medical instrument with optic shape sensing and fiber optic-based oximetry capabilities is shown in accordance with some embodiments. As shown, the system 100 generally includes a console 110 and a stylet assembly 119 communicatively coupled to the console 110. For this embodiment, the stylet assembly 119 includes an elongate probe (e.g., stylet) 120 on its distal end 122 and a console connector 133 on its proximal end 124. It should be understood that the stylet 120 is used herein as being representative of various medical instruments such as a catheter or guidewire. While much of the discussion below describes the integration of an optical fiber into the stylet 120, the same concepts apply to other medical instruments as would be understood by one of ordinary skill in the art.

The console connector 133 enables the stylet assembly 119 to be operably connected to the console 110 via an interconnect 145 including one or more optical fibers 147 (hereinafter, “optical fiber(s)”) and a conductive medium terminated by a single optical/electric connector 146 (or terminated by dual connectors. Herein, the connector 146 is configured to engage (mate) with the console connector 133 to allow for the propagation of light between the console 110 and the stylet assembly 119 as well as the propagation of electrical signals from the stylet 120 to the console 110.

An exemplary implementation of the console 110 includes a processor 160, a memory 165, a display 170 and optical logic 180, although it is appreciated that the console 110 can take one of a variety of forms and may include additional components (e.g., power supplies, ports, interfaces, etc.) that are not directed to aspects of the disclosure. An illustrative example of the console 110 is illustrated in U.S. Pat. No. 10,992,078, the entire contents of which are incorporated by reference herein. The processor 160, with access to the memory 165 (e.g., non-volatile memory or non-transitory, computer-readable medium), is included to control functionality of the console 110 during operation. As shown, the display 170 may be a liquid crystal diode (LCD) display integrated into the console 110 and employed as a user interface to display information to the clinician, especially during a catheter placement procedure (e.g., cardiac catheterization). In another embodiment, the display 170 may be separate from the console 110. Although not shown, a user interface is configured to provide user control of the console 110.

For both of these embodiments, the content depicted by the display 170 may change according to which mode the stylet 120 is configured to operate: optical, tip-location sensor (“TLS”), ECG, or another modality. In TLS mode, the content rendered by the display 170 may constitute a two-dimensional (2D) or three-dimensional (3D) representation of the physical state (e.g., length, shape, form, and/or orientation) of the stylet 120 computed from characteristics of reflected light signals 150 returned to the console 110. The reflected light signals 150 constitute light of a specific spectral width of broadband incident light 155 reflected back to the console 110. According to one embodiment of the disclosure, the reflected light signals 150 may pertain to various discrete portions (e.g., specific spectral widths) of broadband incident light 155 transmitted from and sourced by the optical logic 180, as described below

According to one embodiment of the disclosure, an activation control 126, included on the stylet assembly 119, may be used to set the stylet 120 into a desired operating mode and selectively alter operability of the display 170 by the clinician to assist in medical instrument placement. For example, based on the modality of the stylet 120, the display 170 of the console 110 can be employed for optical modality-based guidance during catheter advancement through the vasculature or TLS modality to determine the physical state (e.g., length, form, shape, orientation, etc.) of the stylet 120. In one embodiment, information from multiple modes, such as optical, TLS or ECG for example, may be displayed concurrently (e.g., at least partially overlapping in time).

Referring still to FIG. 1A, the optical logic 180 is configured to support operability of the stylet assembly 119 and enable the return of information to the console 110, which may be used to determine the physical state associated with the stylet 120 along with monitored electrical signals such as ECG signaling via an electrical signaling logic 181 that supports receipt and processing of the received electrical signals from the stylet 120 (e.g., ports, analog-to-digital conversion logic, etc.). The physical state of the stylet 120 may be based on changes in characteristics of the reflected light signals 150 received at the console 110 from the stylet 120. The characteristics may include shifts in wavelength caused by strain on certain regions of the core fibers integrated within an optical fiber core 135 positioned within or operating as the stylet 120, as shown below. As discussed herein, the optical fiber core 135 may be comprised of core fibers 137₁-137_{M} (M=1 for a single core, and M≥2 for a multi-core), where the core fibers 137₁-137_{M} may collectively be referred to as core fiber(s) 137. Unless otherwise specified or the instant embodiment requires an alternative interpretation, embodiments discussed herein will refer to a multi-core optical fiber 135. From information associated with the reflected light signals 150, the console 110 may determine (through computation or extrapolation of the wavelength shifts) the physical state of the stylet 120, and also that of a catheter 195 configured to receive the stylet 120.

According to one embodiment of the disclosure, as shown in FIG. 1A, the optical logic 180 may include a light source 182 and an optical receiver 184. The light source 182 is configured to transmit the incident light 155 (e.g., broadband) for propagation over the optical fiber(s) 147 included in the interconnect 145, which are optically connected to the multi-core optical fiber core 135 within the stylet 120. In one embodiment, the light source 182 is a tunable swept laser, although other suitable light sources can also be employed in addition to a laser, including semi-coherent light sources, LED light sources, etc.

The optical receiver 184 is configured to: (i) receive returned optical signals, namely reflected light signals 150 received from optical fiber-based reflective gratings (sensors) fabricated within each core fiber of the multi-core optical fiber 135 deployed within the stylet 120, and (ii) translate the reflected light signals 150 into reflection data (from repository 192), namely data in the form of electrical signals representative of the reflected light signals including wavelength shifts caused by strain. The reflected light signals 150 associated with different spectral widths may include reflected light signals 151 provided from sensors positioned in the center core fiber (reference) of the multi-core optical fiber 135 and reflected light signals 152 provided from sensors positioned in the periphery core fibers of the multi-core optical fiber 135, as described below. Herein, the optical receiver 184 may be implemented as a photodetector, such as a positive-intrinsic-negative “PIN” photodiode, avalanche photodiode, or the like.

As shown, both the light source 182 and the optical receiver 184 are operably connected to the processor 160, which governs their operation. Also, the optical receiver 184 is operably coupled to provide the reflection data (from repository 192) to the memory 165 for storage and processing by reflection data classification logic 190. The reflection data classification logic 190 may be configured to: (i) identify which core fibers pertain to which of the received reflection data (from repository 192) and (ii) segregate the reflection data stored with a repository 192 provided from reflected light signals 150 pertaining to similar regions of the stylet 120 or spectral widths into analysis groups. The reflection data for each analysis group is made available to shape sensing logic 194 for analytics.

According to one embodiment of the disclosure, the shape sensing logic 194 is configured to compare wavelength shifts measured by sensors deployed in each periphery core fiber at the same measurement region of the stylet 120 (or same spectral width) to the wavelength shift at a center core fiber of the multi-core optical fiber 135 positioned along central axis and operating as a neutral axis of bending. From these analytics, the shape sensing logic 194 may determine the shape the core fibers have taken in 3D space and may further determine the current physical state of the catheter 195 in 3D space for rendering on the display 170.

According to one embodiment of the disclosure, the shape sensing logic 194 may generate a rendering of the current physical state of the stylet 120 (and potentially the catheter 195), based on heuristics or run-time analytics. For example, the shape sensing logic 194 may be configured in accordance with machine-learning techniques to access a data store (library) with pre-stored data (e.g., images, etc.) pertaining to different regions of the stylet 120 (or catheter 195) in which reflected light from core fibers have previously experienced similar or identical wavelength shifts. From the pre-stored data, the current physical state of the stylet 120 (or catheter 195) may be rendered. Alternatively, as another example, the shape sensing logic 194 may be configured to determine, during run-time, changes in the physical state of each region of the multi-core optical fiber 135 based on at least: (i) resultant wavelength shifts experienced by different core fibers within the optical fiber 135, and (ii) the relationship of these wavelength shifts generated by sensors positioned along different periphery core fibers at the same cross-sectional region of the multi-core optical fiber 135 to the wavelength shift generated by a sensor of the center core fiber at the same cross-sectional region. It is contemplated that other processes and procedures may be performed to utilize the wavelength shifts as measured by sensors along each of the core fibers within the multi-core optical fiber 135 to render appropriate changes in the physical state of the stylet 120 (and/or catheter 195), especially to enable guidance of the stylet 120, when positioned at a distal tip of the catheter 195, within the vasculature of the patient and at a desired destination within the body.

The console 110 may further include electrical signaling logic 181, which is positioned to receive one or more electrical signals from the stylet 120. The stylet 120 is configured to support both optical connectivity as well as electrical connectivity. The electrical signaling logic 181 receives the electrical signals (e.g., ECG signals) from the stylet 120 via the conductive medium. The electrical signals may be processed by electrical signal logic 196, executed by the processor 160, to determine ECG waveforms for display.

Referring to FIG. 1B, an alternative exemplary embodiment of a medical instrument monitoring system 100 is shown. Herein, the medical instrument monitoring system 100 features a console 110 and a medical instrument 130 communicatively coupled to the console 110. For this embodiment, the medical instrument 130 corresponds to a catheter, which features an integrated tubing with two or more lumen extending between a proximal end 131 and a distal end 132 of the integrated tubing. The integrated tubing (sometimes referred to as “catheter tubing”) is in communication with one or more extension legs 140 via a bifurcation hub 142. An optical-based catheter connector 144 may be included on a proximal end of at least one of the extension legs 140 to enable the catheter 130 to operably connect to the console 110 via an interconnect 145 or another suitable component. Herein, the interconnect 145 may include a connector 146 that, when coupled to the optical-based catheter connector 144, establishes optical connectivity between one or more optical fibers 147 (hereinafter, “optical fiber(s)”) included as part of the interconnect 145 and core fibers 137 deployed within the catheter 130 and integrated into the tubing. Alternatively, a different combination of connectors, including one or more adapters, may be used to optically connect the optical fiber(s) 147 to the core fibers 137 within the catheter 130. The core fibers 137 deployed within the catheter 130 as illustrated in FIG. 1B include the same characteristics and perform the same functionalities as the core fibers 137 deployed within the stylet 120 of FIG. 1A.

The optical logic 180 is configured to support graphical rendering of the catheter 130, most notably the integrated tubing of the catheter 130, based on characteristics of the reflected light signals 150 received from the catheter 130. The characteristics may include shifts in wavelength caused by strain on certain regions of the core fibers 137 integrated within (or along) a wall of the integrated tubing, which may be used to determine (through computation or extrapolation of the wavelength shifts) the physical state of the catheter 130, notably its integrated tubing or a portion of the integrated tubing such as a tip or distal end of the tubing to read fluctuations (real-time movement) of the tip (or distal end).

More specifically, the optical logic 180 includes a light source 182. The light source 182 is configured to transmit the broadband incident light 155 for propagation over the optical fiber(s) 147 included in the interconnect 145, which are optically connected to multiple core fibers 137 within the catheter tubing. Herein, the optical receiver 184 is configured to: (i) receive returned optical signals, namely reflected light signals 150 received from optical fiber-based reflective gratings (sensors) fabricated within each of the core fibers 137 deployed within the catheter 130, and (ii) translate the reflected light signals 150 into reflection data (from repository 192), namely data in the form of electrical signals representative of the reflected light signals including wavelength shifts caused by strain. The reflected light signals 150 associated with different spectral widths include reflected light signals 151 provided from sensors positioned in the center core fiber (reference) of the catheter 130 and reflected light signals 152 provided from sensors positioned in the outer core fibers of the catheter 130, as described below.

As noted above, the shape sensing logic 194 is configured to compare wavelength shifts measured by sensors deployed in each outer core fiber at the same measurement region of the catheter (or same spectral width) to the wavelength shift at the center core fiber positioned along central axis and operating as a neutral axis of bending. From these analytics, the shape sensing logic 190 may determine the shape the core fibers have taken in 3D space and may further determine the current physical state of the catheter 130 in 3D space for rendering on the display 170.

According to one embodiment of the disclosure, the shape sensing logic 194 may generate a rendering of the current physical state of the catheter 130, especially the integrated tubing, based on heuristics or run-time analytics. For example, the shape sensing logic 194 may be configured in accordance with machine-learning techniques to access a data store (library) with pre-stored data (e.g., images, etc.) pertaining to different regions of the catheter 130 in which the core fibers 137 experienced similar or identical wavelength shifts. From the pre-stored data, the current physical state of the catheter 130 may be rendered. Alternatively, as another example, the shape sensing logic 194 may be configured to determine, during run-time, changes in the physical state of each region of the catheter 130, notably the tubing, based on at least (i) resultant wavelength shifts experienced by the core fibers 137 and (ii) the relationship of these wavelength shifts generated by sensors positioned along different outer core fibers at the same cross-sectional region of the catheter 130 to the wavelength shift generated by a sensor of the center core fiber at the same cross-sectional region. It is contemplated that other processes and procedures may be performed to utilize the wavelength shifts as measured by sensors along each of the core fibers 137 to render appropriate changes in the physical state of the catheter 130.

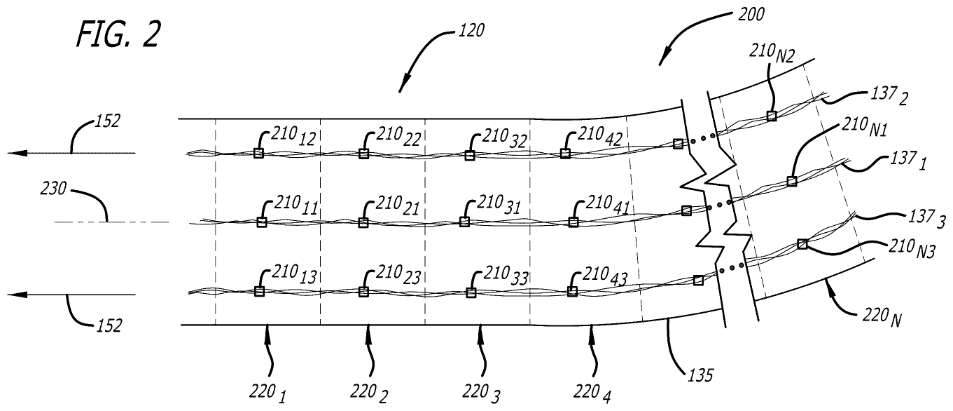

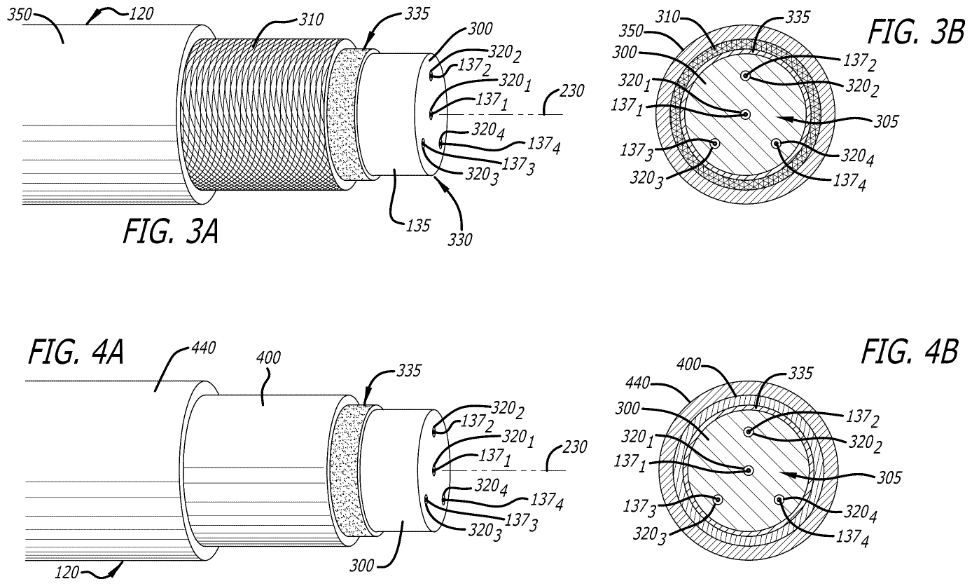

Referring to FIG. 2, an exemplary embodiment of a structure of a section of the multi-core optical fiber included within the stylet 120 of FIG. 1A is shown in accordance with some embodiments. The multi-core optical fiber section 200 of the multi-core optical fiber 135 depicts certain core fibers 137₁-137_{M} (M≥2, M=4 as shown, see FIG. 3A) along with the spatial relationship between sensors (e.g., reflective gratings) 210₁₁-210_{NM} (N≥2; M≥2) present within the core fibers 137₁-137_{M}, respectively. As noted above, the core fibers 137₁-137_{M} may be collectively referred to as “the core fibers 137.”

As shown, the section 200 is subdivided into a plurality of cross-sectional regions 220₁-220_{N}, where each cross-sectional region 220₁-220_{N} corresponds to reflective gratings 210₁₁-210₁₄ . . . 210_{N1}-210_{N4}. Some or all of the cross-sectional regions 220₁ . . . 220_{N} may be static (e.g., prescribed length) or may be dynamic (e.g., vary in size among the regions 220₁ . . . 220_{N}). A first core fiber 137₁ is positioned substantially along a center (neutral) axis 230 while core fiber 137₂ may be oriented within the cladding of the multi-core optical fiber 135, from a cross-sectional, front-facing perspective, to be position on “top” the first core fiber 137₁. In this deployment, the core fibers 137₃ and 137₄ may be positioned “bottom left” and “bottom right” of the first core fiber 137₁. As examples, FIGS. 3A-4B provides illustrations of such.

Referencing the first core fiber 137₁ as an illustrative example, when the stylet 120 is operative, each of the reflective gratings 210₁-210_{N} reflects light for a different spectral width. As shown, each of the gratings 210₁ᵢ-210_{Ni} (1≤i≤M) is associated with a different, specific spectral width, which would be represented by different center frequencies of ƒ₁ . . . ƒ_{N}, where neighboring spectral widths reflected by neighboring gratings are non-overlapping according to one embodiment of the disclosure.

Herein, positioned in different core fibers 137₂-137₃ but along at the same cross-sectional regions 220-220_{N} of the multi-core optical fiber 135, the gratings 210₁₂-210_{N2} and 210₁₃-210_{N3} are configured to reflect incoming light at same (or substantially similar) center frequency. As a result, the reflected light returns information that allows for a determination of the physical state of the optical fibers 137 (and the stylet 120) based on wavelength shifts measured from the returned, reflected light. In particular, strain (e.g., compression or tension) applied to the multi-core optical fiber 135 (e.g., at least core fibers 137₂-137₃) results in wavelength shifts associated with the returned, reflected light. Based on different locations, the core fibers 137₁-137₄ experience different types and degree of strain based on angular path changes as the stylet 120 advances in the patient.

For example, with respect to the multi-core optical fiber section 200 of FIG. 2, in response to angular (e.g., radial) movement of the stylet 120 is in the left-veering direction, the fourth core fiber 137₄ (see FIG. 3A) of the multi-core optical fiber 135 with the shortest radius during movement (e.g., core fiber closest to a direction of angular change) would exhibit compression (e.g., forces to shorten length). At the same time, the third core fiber 137₃ with the longest radius during movement (e.g., core fiber furthest from the direction of angular change) would exhibit tension (e.g., forces to increase length). As these forces are different and unequal, the reflected light from reflective gratings 210_{N2} and 210_{N3} associated with the core fiber 137₂ and 137₃ will exhibit different changes in wavelength. The differences in wavelength shift of the reflected light signals 150 can be used to extrapolate the physical configuration of the stylet 120 by determining the degrees of wavelength change caused by compression/tension for each of the periphery fibers (e.g., the second core fiber 137₂ and the third core fiber 137₃) in comparison to the wavelength of the reference core fiber (e.g., first core fiber 137₁) located along the neutral axis 230 of the multi-core optical fiber 135. These degrees of wavelength change may be used to extrapolate the physical state of the stylet 120. The reflected light signals 150 are reflected back to the console 110 via individual paths over a particular core fiber 137₁-137_{M}.

Referring to FIG. 3A, a first exemplary embodiment of the stylet of FIG. 1A supporting both an optical and electrical signaling is shown in accordance with some embodiments. Herein, the stylet 120 features a centrally located multi-core optical fiber 135, which includes a cladding 300 and a plurality of core fibers 137₁-137_{M} (M≥2; M=4) residing within a corresponding plurality of lumens 320₁-320_{M}. While the multi-core optical fiber 135 is illustrated within four (4) core fibers 137₁-137₄, a greater number of core fibers 137₁-137_{M} (M>4) may be deployed to provide a more detailed three-dimensional sensing of the physical state (e.g., shape, etc.) of the multi-core optical fiber 135 and the stylet 120 deploying the optical fiber 135.

For this embodiment of the disclosure, the multi-core optical fiber 135 is encapsulated within a concentric braided tubing 310 positioned over a low coefficient of friction layer 335. The braided tubing 310 may feature a “mesh” construction, in which the spacing between the intersecting conductive elements is selected based on the degree of rigidity desired for the stylet 120, as a greater spacing may provide a lesser rigidity, and thereby, a more pliable stylet 120.

According to this embodiment of the disclosure, as shown in FIGS. 3A-3B, the core fibers 137₁-137₄ include (i) a central core fiber 137₁ and (ii) a plurality of periphery core fibers 137₂-137₄, which are maintained within lumens 320₁-320₄ formed in the cladding 300. According to one embodiment of the disclosure, one or more of the lumen 320₁-320₄ may be configured with a diameter sized to be greater than the diameter of the core fibers 137₁-137₄. By avoiding a majority of the surface area of the core fibers 137₁-137₄ from being in direct physical contact with a wall surface of the lumens 320₁-320₄, the wavelength changes to the incident light are caused by angular deviations in the multi-core optical fiber 135 thereby reducing influence of compression and tension forces being applied to the walls of the lumens 320₁-320_{M}, not the core fibers 137₁-137_{M} themselves.

As further shown in FIGS. 3A-3B, the core fibers 137₁-137₄ may include central core fiber 137₁ residing within a first lumen 320₁ formed along the first neutral axis 230 and a plurality of core fibers 137₂-137₄ residing within lumens 320₂-320₄ each formed within different areas of the cladding 300 radiating from the first neutral axis 230. In general, the core fibers 137₂-137₄, exclusive of the central core fiber 137₁, may be positioned at different areas within a cross-sectional area 305 of the cladding 300 to provide sufficient separation to enable three-dimensional sensing of the multi-core optical fiber 135 based on changes in wavelength of incident light propagating through the core fibers 137₂-137₄ and reflected back to the console for analysis.

For example, where the cladding 300 features a circular cross-sectional area 305 as shown in FIG. 3B, the core fibers 137₂-137₄ may be positioned substantially equidistant from each other as measured along a perimeter of the cladding 300, such as at “top” (12 o'clock), “bottom-left” (8 o'clock) and “bottom-right” (4 o'clock) locations as shown. Hence, in general terms, the core fibers 137₂-137₄ may be positioned within different segments of the cross-sectional area 305. Where the cross-sectional area 305 of the cladding 300 has a distal tip 330 and features a polygon cross-sectional shape (e.g., triangular, square, rectangular, pentagon, hexagon, octagon, etc.), the central core fiber 137₁ may be located at or near a center of the polygon shape, while the remaining core fibers 137₂-137_{M} may be located proximate to angles between intersecting sides of the polygon shape.

Referring still to FIGS. 3A-3B, operating as the conductive medium for the stylet 120, the braided tubing 310 provides mechanical integrity to the multi-core optical fiber 135 and operates as a conductive pathway for electrical signals. For example, the braided tubing 310 may be exposed to a distal tip of the stylet 120. The cladding 300 and the braided tubing 310, which is positioned concentrically surrounding a circumference of the cladding 300, are contained within the same insulating layer 350. The insulating layer 350 may be a sheath or conduit made of protective, insulating (e.g., non-conductive) material that encapsulates both for the cladding 300 and the braided tubing 310, as shown.

Referring to FIG. 4A, a second exemplary embodiment of the stylet of FIG. 1B is shown in accordance with some embodiments. Referring now to FIG. 4A, a second exemplary embodiment of the stylet 120 of FIG. 1B supporting both an optical and electrical signaling is shown. Herein, the stylet 120 features the multi-core optical fiber 135 described above and shown in FIG. 3A, which includes the cladding 300 and the first plurality of core fibers 137₁-137_{M}(M≥3; M=4 for embodiment) residing within the corresponding plurality of lumens 320₁-320_{M}. For this embodiment of the disclosure, the multi-core optical fiber 135 includes the central core fiber 137₁ residing within the first lumen 320₁ formed along the first neutral axis 230 and the second plurality of core fibers 137₂-137₄ residing within corresponding lumens 320₂-320₄ positioned in different segments within the cross-sectional area 305 of the cladding 300. Herein, the multi-core optical fiber 135 is encapsulated within a conductive tubing 400. The conductive tubing 400 may feature a “hollow” conductive cylindrical member concentrically encapsulating the multi-core optical fiber 135.

Referring to FIGS. 4A-4B, operating as a conductive medium for the stylet 120 in the transfer of electrical signals (e.g., ECG signals) to the console, the conductive tubing 400 may be exposed up to a tip 410 of the stylet 120. For this embodiment of the disclosure, a conductive epoxy 420 (e.g., metal-based epoxy such as a silver epoxy) may be affixed to the tip 410 and similarly joined with a termination/connection point created at a proximal end 430 of the stylet 120. The cladding 300 and the conductive tubing 400, which is positioned concentrically surrounding a circumference of the cladding 300, are contained within the same insulating layer 440. The insulating layer 440 may be a protective conduit encapsulating both for the cladding 300 and the conductive tubing 400, as shown.

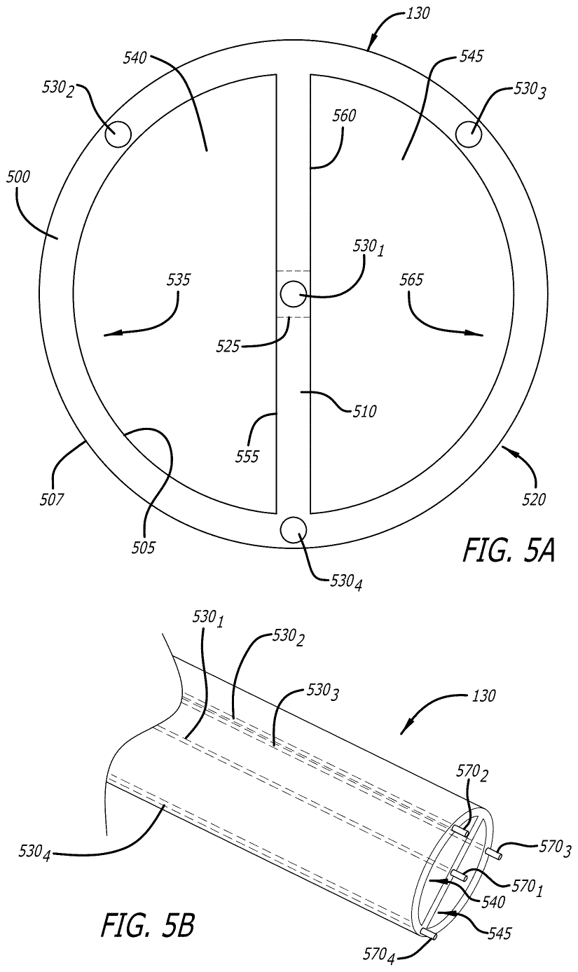

Referring to FIG. 5A, an elevation view of a first illustrative embodiment of a catheter including integrated tubing, a diametrically disposed septum, and micro-lumens formed within the tubing and septum is shown in accordance with some embodiments. Herein, the catheter 130 includes integrated tubing, the diametrically disposed septum 510, and the plurality of micro-lumens 530₁-530₄ which, for this embodiment, are fabricated to reside within the wall 500 of the integrated tubing of the catheter 130 and within the septum 510. In particular, the septum 510 separates a single lumen, formed by the inner surface 505 of the wall 500 of the catheter 130, into multiple lumen, namely two lumens 540 and 545 as shown. Herein, the first lumen 540 is formed between a first arc-shaped portion 535 of the inner surface 505 of the wall 500 forming the catheter 130 and a first outer surface 555 of the septum 510 extending longitudinally within the catheter 130. The second lumen 545 is formed between a second arc-shaped portion 565 of the inner surface 505 of the wall 500 forming the catheter 130 and a second outer surfaces 560 of the septum 510.

According to one embodiment of the disclosure, the two lumens 540 and 545 have approximately the same volume. However, the septum 510 need not separate the tubing into two equal lumens. For example, instead of the septum 510 extending vertically (12 o'clock to 6 o'clock) from a front-facing, cross-sectional perspective of the tubing, the septum 510 could extend horizontally (3 o'clock to 9 o'clock), diagonally (1 o'clock to 7 o'clock; 10 o'clock to 4 o'clock) or angularly (2 o'clock to 10 o'clock). In the later configuration, each of the lumens 540 and 545 of the catheter 130 would have a different volume.

With respect to the plurality of micro-lumens 530₁-530₄, the first micro-lumen 530₁ is fabricated within the septum 510 at or near the cross-sectional center 525 of the integrated tubing. For this embodiment, three micro-lumens 530₂-530₄ are fabricated to reside within the wall 500 of the catheter 130. In particular, a second micro-lumen 530₂ is fabricated within the wall 500 of the catheter 130, namely between the inner surface 505 and outer surface 507 of the first arc-shaped portion 535 of the wall 500. Similarly, the third micro-lumen 530₃ is also fabricated within the wall 500 of the catheter 130, namely between the inner and outer surfaces 505/507 of the second arc-shaped portion 555 of the wall 500. The fourth micro-lumen 530₄ is also fabricated within the inner and outer surfaces 505/507 of the wall 500 that are aligned with the septum 510.

According to one embodiment of the disclosure, as shown in FIG. 5A, the micro-lumens 530₂-530₄ are positioned in accordance with a “top-left” (10 o'clock), “top-right” (2 o'clock) and “bottom” (6 o'clock) layout from a front-facing, cross-sectional perspective. Of course, the micro-lumens 530₂-530₄ may be positioned differently, provided that the micro-lumens 530₂-530₄ are spatially separated along the circumference 520 of the catheter 130 to ensure a more robust collection of reflected light signals from the outer core fibers 570₂-570₄ when installed. For example, two or more of micro-lumens (e.g., micro-lumens 530₂ and 530₄) may be positioned at different quadrants along the circumference 520 of the catheter wall 500.

Referring to FIG. 5B, a perspective view of the first illustrative embodiment of the catheter of FIG. 5A including core fibers installed within the micro-lumens is shown in accordance with some embodiments. According to one embodiment of the disclosure, the second plurality of micro-lumens 530₂-530₄ are sized to retain corresponding outer core fibers 570₂-570₄, where the diameter of each of the second plurality of micro-lumens 530₂-530₄ may be sized just larger than the diameters of the outer core fibers 570₂-570₄. The size differences between a diameter of a single core fiber and a diameter of any of the micro-lumen 530₁-530₄ may range between 0.001 micrometers (μm) and 1000μm, for example. As a result, the cross-sectional areas of the outer core fibers 570₂-570₄ would be less than the cross-sectional areas of the corresponding micro-lumens 530₂-530₄. A “larger” micro-lumen (e.g., micro-lumen 530₂) may better isolate external strain being applied to the outer core fiber 570₂ from strain directly applied to the catheter 130 itself. Similarly, the first micro-lumen 530₁ may be sized to retain the center core fiber 570₁, where the diameter of the first micro-lumen 530₁ may be sized just larger than the diameter of the center core fiber 570₁.

As an alternative embodiment of the disclosure, one or more of the micro-lumens 530₁-530₄ may be sized with a diameter that exceeds the diameter of the corresponding one or more core fibers 570₁-570₄. However, at least one of the micro-lumens 530₁-530₄ is sized to fixedly retain their corresponding core fiber (e.g., core fiber retained with no spacing between its lateral surface and the interior wall surface of its corresponding micro-lumen). As yet another alternative embodiment of the disclosure, all the micro-lumens 530₁-530₄ are sized with a diameter to fixedly retain the core fibers 570₁-570₄.

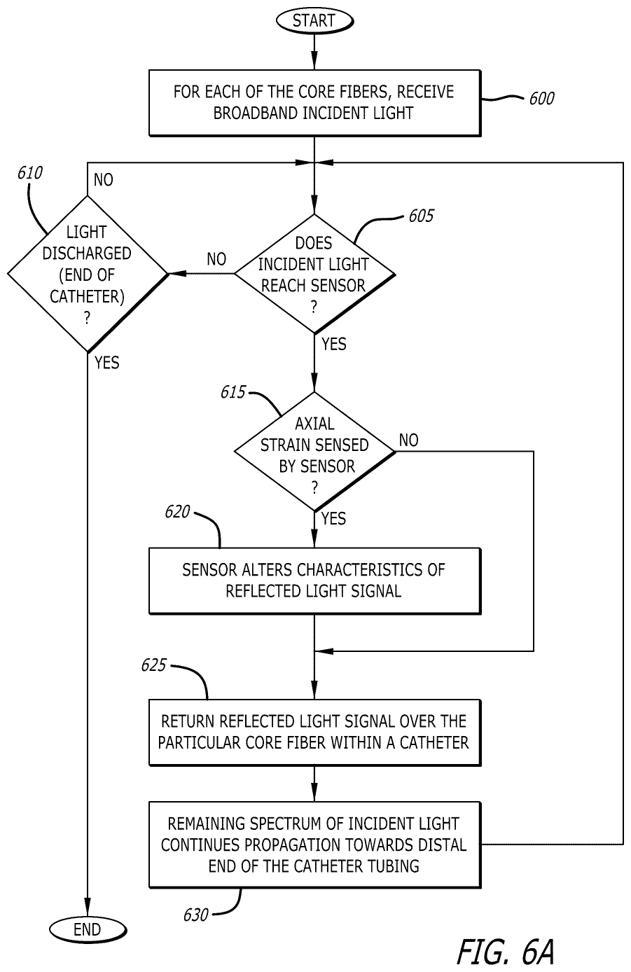

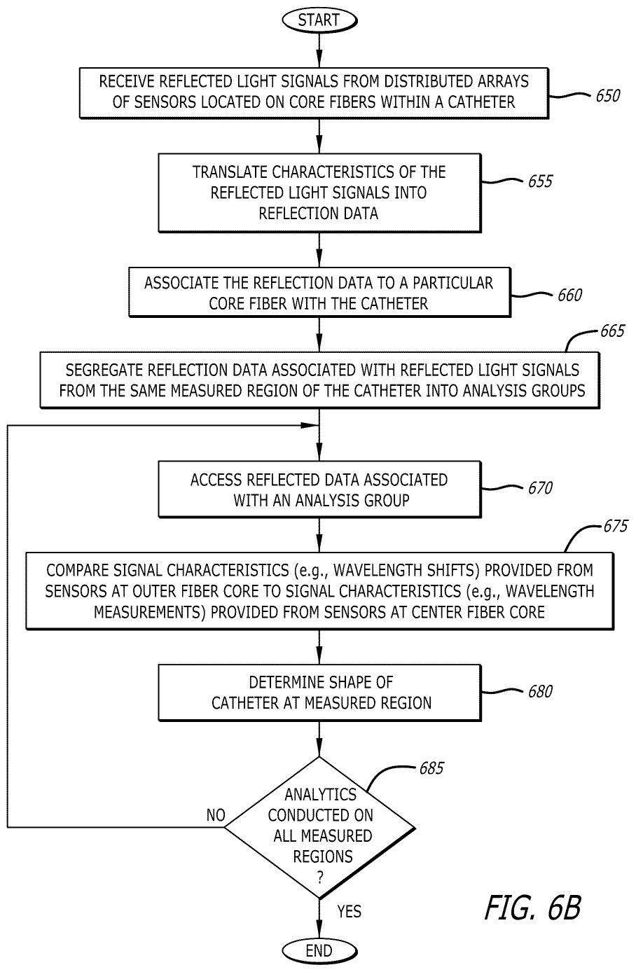

Referring to FIGS. 6A-6B, flowcharts of methods of operations conducted by the medical instrument monitoring system of FIGS. 1A-1B to achieve optic 3D shape sensing are shown in accordance with some embodiments. Herein, the catheter includes at least one septum spanning across a diameter of the tubing wall and continuing longitudinally to subdivide the tubing wall. The medial portion of the septum is fabricated with a first micro-lumen, where the first micro-lumen is coaxial with the central axis of the catheter tubing. The first micro-lumen is configured to retain a center core fiber. Two or more micro-lumen, other than the first micro-lumen, are positioned at different locations circumferentially spaced along the wall of the catheter tubing. For example, two or more of the second plurality of micro-lumens may be positioned at different quadrants along the circumference of the catheter wall.

Furthermore, each core fiber includes a plurality of sensors spatially distributed along its length between at least the proximal and distal ends of the catheter tubing. This array of sensors is distributed to position sensors at different regions of the core fiber to enable distributed measurements of strain throughout the entire length or a selected portion of the catheter tubing. These distributed measurements may be conveyed through reflected light of different spectral widths (e.g., specific wavelength or specific wavelength ranges) that undergoes certain wavelength shifts based on the type and degree of strain.

According to one embodiment of the disclosure, as shown in FIG. 6A, for each core fiber, broadband incident light is supplied to propagate through a particular core fiber (block 600). Unless discharged, upon the incident light reaching a sensor of a distributed array of sensors measuring strain on a particular core fiber, light of a prescribed spectral width associated with the first sensor is to be reflected back to an optical receiver within a console (blocks 605-610). Herein, the sensor alters characteristics of the reflected light signal to identify the type and degree of strain on the particular core fiber as measured by the first sensor (blocks 615-620). According to one embodiment of the disclosure, the alteration in characteristics of the reflected light signal may signify a change (shift) in the wavelength of the reflected light signal from the wavelength of the incident light signal associated with the prescribed spectral width. The sensor returns the reflected light signal over the core fiber and the remaining spectrum of the incident light continues propagation through the core fiber toward a distal end of the catheter tubing (blocks 625-630). The remaining spectrum of the incident light may encounter other sensors of the distributed array of sensors, where each of these sensors would operate as set forth in blocks 605-630 until the last sensor of the distributed array of sensors returns the reflected light signal associated with its assigned spectral width and the remaining spectrum is discharged as illumination.

Referring now to FIG. 6B, during operation, multiple reflected light signals are returned to the console from each of the plurality of core fibers residing within the corresponding plurality of micro-lumens formed within a catheter, such as the catheter of FIG. 1B. In particular, the optical receiver receives reflected light signals from the distributed arrays of sensors located on the center core fiber and the outer core fibers and translates the reflected light signals into reflection data, namely electrical signals representative of the reflected light signals including wavelength shifts caused by strain (blocks 650-655). The reflection data classification logic is configured to identify which core fibers pertain to which reflection data and segregate reflection data provided from reflected light signals pertaining to a particular measurement region (or similar spectral width) into analysis groups (block 660-665).

Each analysis group of reflection data is provided to shape sensing logic for analytics (block 670). Herein, the shape sensing logic compares wavelength shifts at each outer core fiber with the wavelength shift at the center core fiber positioned along central axis and operating as a neutral axis of bending (block 675). From this analytics, on all analytic groups (e.g., reflected light signals from sensors in all or most of the core fibers), the shape sensing logic may determine the shape the core fibers have taken in three-dimensional space, from which the shape sensing logic can determine the current physical state of the catheter in three-dimension space (blocks 680-685).

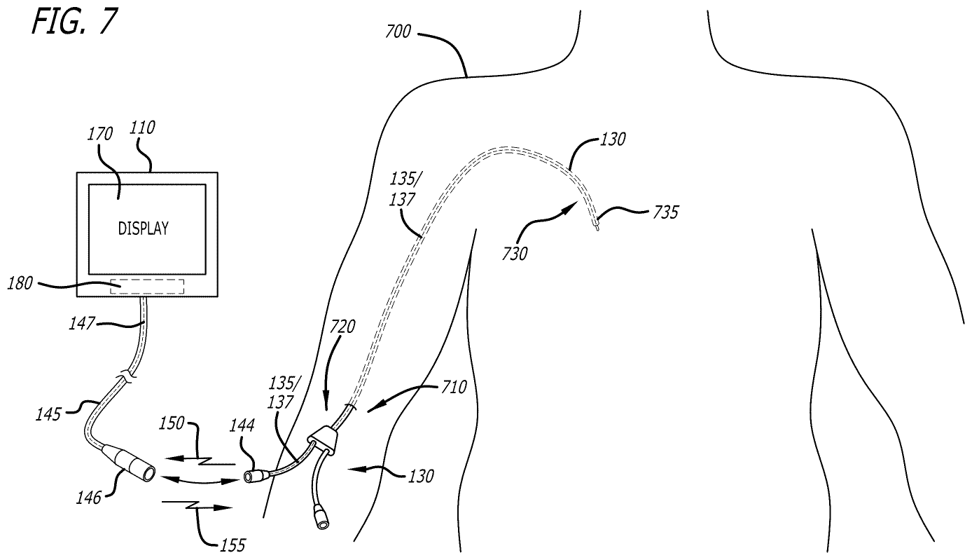

Referring to FIG. 7, an exemplary embodiment of the medical instrument monitoring system of FIG. 1B during operation and insertion of the catheter into a patient are shown in accordance with some embodiments. Herein, the catheter 130 generally includes the integrated tubing of the catheter 130 with a proximal portion 720 that generally remains exterior to the patient 700 and a distal portion 730 that generally resides within the patient vasculature after placement is complete. The (integrated) catheter tubing of the catheter 130 may be advanced to a desired position within the patient vasculature such as a distal end (or tip) 735 of the catheter tubing of the catheter 130 is proximate the patient's heart, such as in the lower one-third (⅓) portion of the Superior Vena Cava (“SVC”) for example. In some embodiments, various instruments may be disposed at the distal end 735 of the catheter 130 to measure pressure of blood in a certain heart chamber and in the blood vessels, view an interior of blood vessels, or the like. In alternative embodiments, such as those that utilize the stylet assembly of FIG. 1A and the catheter 195, such instruments may be disposed at a distal end of the stylet 120.

During advancement through a patient vasculature, the catheter tubing of the catheter 130 receives broadband incident light 155 from the console 110 via optical fiber(s) 147 within the interconnect 145, where the incident light 155 propagates along the core fibers 137 of the multi-core optical fiber 135 within the catheter tubing of the catheter 130. According to one embodiment of the disclosure, the connector 146 of the interconnect 145 terminating the optical fiber(s) 147 may be coupled to the optical-based catheter connector 144, which may be configured to terminate the core fibers 137 deployed within the catheter 130. Such coupling optically connects the core fibers 137 of the catheter 130 with the optical fiber(s) 147 within the interconnect 145. The optical connectivity is needed to propagate the incident light 155 to the core fibers 137 and return the reflected light signals 150 to the optical logic 180 within the console 110 over the interconnect 145. As described below in detail, the physical state of the catheter 130 may be ascertained based on analytics of the wavelength shifts of the reflected light signals 150.

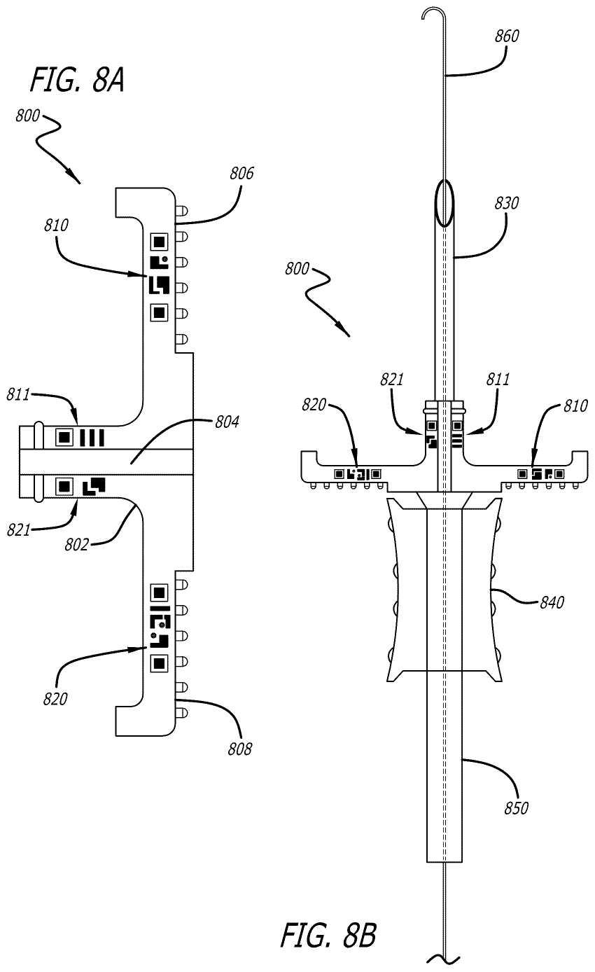

Referring now to FIG. 8A, an example introducer including a set of markings disposed thereon is shown in accordance with some embodiments. FIG. 8A illustrates an introducer 800 that includes a body 802 having a channel 804 extending therethrough and, optionally, arms 806, 808 that extend outwardly from the body 802. The introducer 800 may include one or more markings 810, 811, 820, 821 that are configured to enable identification of the introducer 800. As shown, the markings 810, 811, 820, 821 may be located on the arms 806, 808, and body 802. In other embodiments, the introducer 800 may only include a single marking. In further embodiments, the introducer 800 may include one or more markings 810, 811, 820, 821 on the body 802. It should be understood that the markings 810, 811, 820, 821 are merely illustrative markings and the disclosure is intended to cover other visual markings such as a pattern of lines or other geometric figures, an alphanumeric identifier, etc. From such markings, position and orientation may be determined.

The introducer 800 may be configured for use in a vascular access procedure, such as placing a central line, a catheter, or performing angiography. The introducer 800 may be a small, sturdy medical instrument configured to assist a medical professional safely and easily insert other medical instruments such as a catheter, a stylet, a guidewire, etc., into a blood vessel within a patient vasculature. For example, the channel 804 creates a stable channel for passing such medical instruments in and out of the blood vessel.

As will be discussed in further detail below, the markings 810, 811, 820, 821 may be used by an extended reality (XR) headset to identify the introducer 800. The identification may be performed through object detection performed by XR visualization logic and includes analyzing frames of a set of frames captured by the XR headset and identifying specific patterns or objects in an image. The XR headset may include external (or outwardly) facing cameras that capture image frames (video) in real-time. The XR visualization logic estimates a position of the introducer 800 in the environment (a medical procedure environment), which results in assigning the introducer 800 three-dimensional (3D) coordinates (e.g., x, z) in a 3D map of the medical procedure environment automatically generated by the XR visualization logic.

Referring to FIG. 8B, the example introducer of FIG. 8A coupled with a needle having a medical instrument threaded therethrough is shown in accordance with some embodiments. The introducer 800 is shown coupled to a finger grip 840 and a hub 850. Additionally, a needle 830 is disposed within the channel 804. Further, a medical instrument that is to be propagated through a patient vasculature is shown to be threaded through a lumen of each of the hub 850, the finger grip, 840, and the needle 830. The medical instrument 860 may be, for example, a guidewire, a stylet, a catheter, etc.

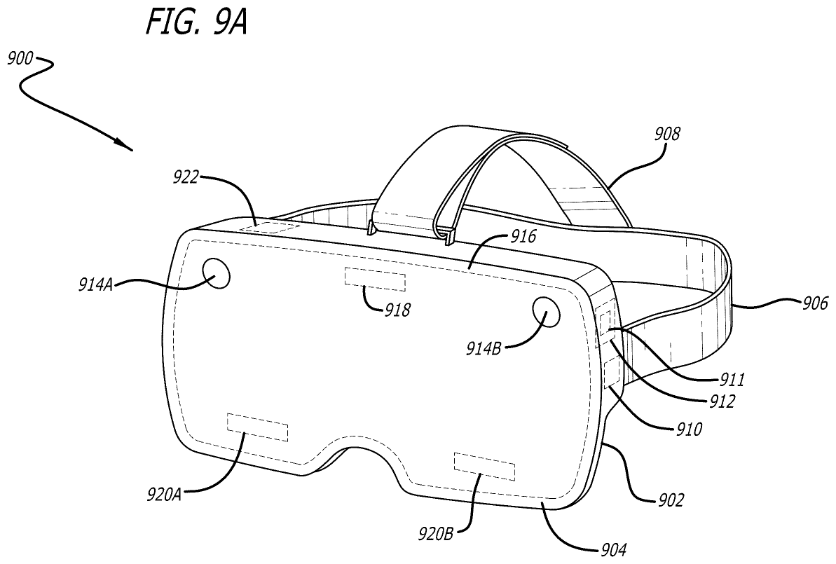

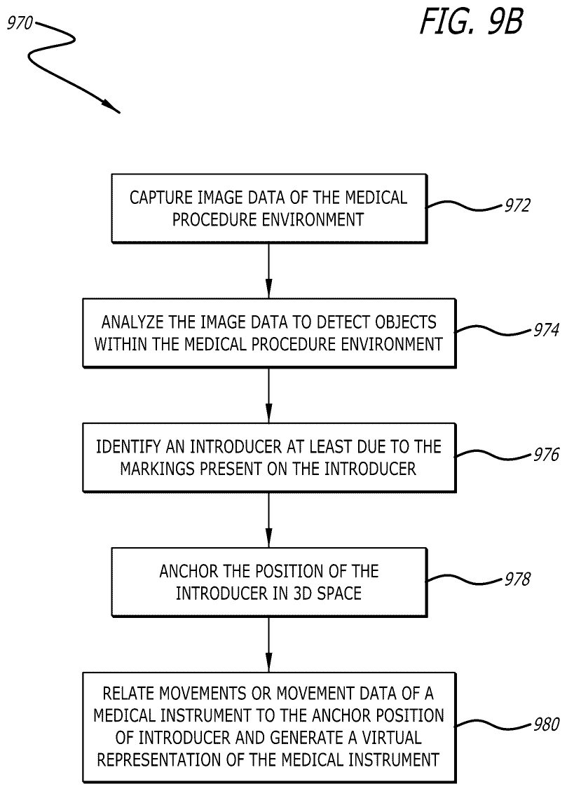

Referring now to FIG. 9A, an illustrative example of an XR headset is shown in accordance with some embodiments. The XR headset 900 includes a body 902 and a lens 904 with a lower head strap 906 and an upper head strap 908 each coupled to the body 902 and configured to securely couple the headset to an individual's head. FIG. 9A is merely illustrative of a VR headset and any design elements shown are not intended to limit the scope of the disclosure. The body 902 houses certain components for XR processing and image generation including one or more processors 910, non-transitory computer-readable medium 912 having stored thereon executable instructions (logic, namely XR logic 913), projectors/LEDs 914A, 914B configured to display an image to the individual wearing the XR headset 900, waveguides 916, a tracking system 918, eye tracking LEDs 920A, 920B, and a battery 922. The waveguides 916 may be formed of thin pieces of plastic or glass in a stacked orientation and are configured to transmit and bend light through total internal reflection (TIR), which reflects light between the inner and outer edges of the layers of the waveguides resulting in a display that visually combines virtual (digital) and real-world images together. The waveguides 916 may be formed of mirror arrays, prisms, metasurfaces, or gratings. In embodiments, the waveguides 916 include six waveguide layers comprising two layers for each color channel (red, green, blue).

The tracking system 918 includes one or more forward-facing cameras, inertial measurement units (accelerometer, gyroscope), and depth sensors. In combination with the XR logic 913, the tracking system 918 is configured to track user movements and orientation relative to the user's environment across three axes (x, y, z). Typically, six types of user movements are tracked including: movements left and right along the x-axis (surge), movements up and down along the y-axis (heave); movements front and back along the z-axis (sway); rotation along the vertical axis (i.e., turn the head left and right) (yaw); rotation along the side-to-side axis (i.e., look up and down) (pitch); and rotation along the front-to-back axis (i.e., tilt the head towards the shoulders) (roll).

In combination with the XR logic 913, the eye tracking system may include infrared (IR) emitters that emit IR light, IR cameras that capture images of the user's pupils and corneal reflections, and logic, that when executed by the processors 910, calculate a gaze direction of the user, which enables the XR logic 913 to render different portions of displayed graphics in the appropriate focus (e.g., in focus or greater resolution where the user is looking), tracking eye moments to enable selection of items using sight, etc. It is noted that movements of each eye are tracked separately and then analyzed together by the XR logic 913 to determine gaze direction, selection of items displayed by the XR headset 900, etc.

FIG. 9B is a flowchart of a set of operations conducted by the XR headset 900 in identifying the introducer 800 and determining an anchor position within a medical procedure environment in accordance with some embodiments. The process 970 can be implemented, for example, by a computing device, e.g., an XR headset 900, that comprises a processor and a non-transitory computer-readable medium. The non-transitory computer readable medium may store instructions that, when executed by the processor, can cause the processor to perform the operations of the illustrated process 970.

Each block illustrated in FIG. 9B represents an operation in the process 970 performed by, for example, the XR headset 900 of FIG. 9A. It should be understood that not every operation illustrated in FIG. 9B is required. In fact, certain operations may be optional to complete aspects of the process 970. The process 970 begins with components of the XR headset 900 capture image data of the medical procedure environment (block 972). The image data may be a video stream that is continuously recording in real-time in order to track the user's movements within the medical procedure environment. The XR logic 913 analyzes the image data to detect objects (block 974). The analysis may include the use of computer vision techniques and process the image data using object detection algorithms that include deep learning machine learning models such as YOLO, SSD, etc., which may include pattern, edge, color, shape, and/or texture detection and matching of such to historical image data that was used to train the models or algorithms. The XR headset 900 identifies the introducer 800 of FIGS. 8A-8B at least due to the markings 810, 811, 820, 821 (block 976). The XR logic 913 may identify the introducer 800 by drawing a bounding box around the introducer 800, labeling the introducer 800 (e.g., as an introducer), and estimating its position within a 3D space relative to the user. The XR headset 900 anchors the position of the introducer 800 in 3D space (978). The anchoring of the introducer 800 includes recording and maintaining its position within a virtual map generated by the XR logic 913 of the medical procedure environment.

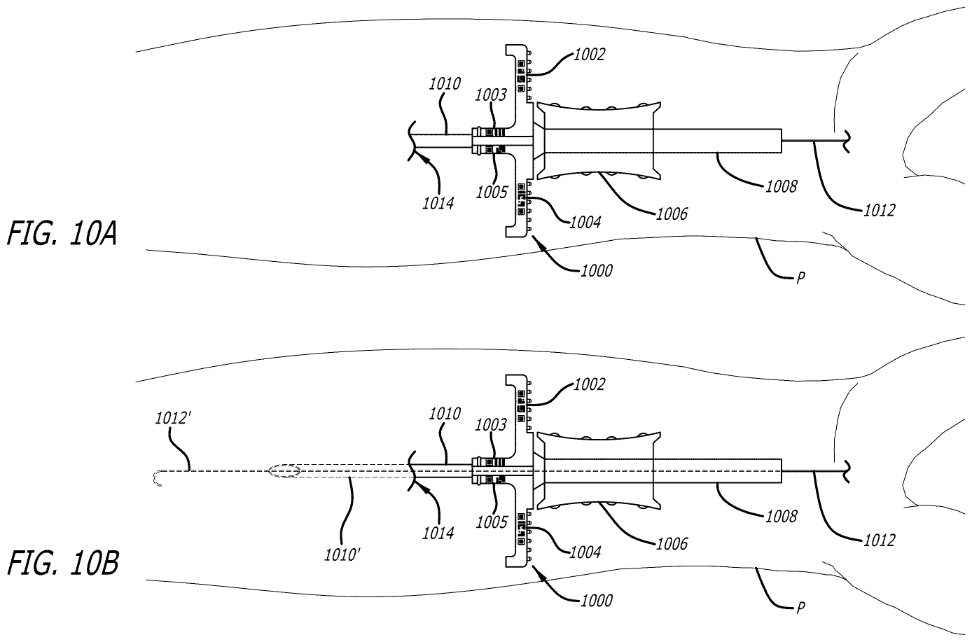

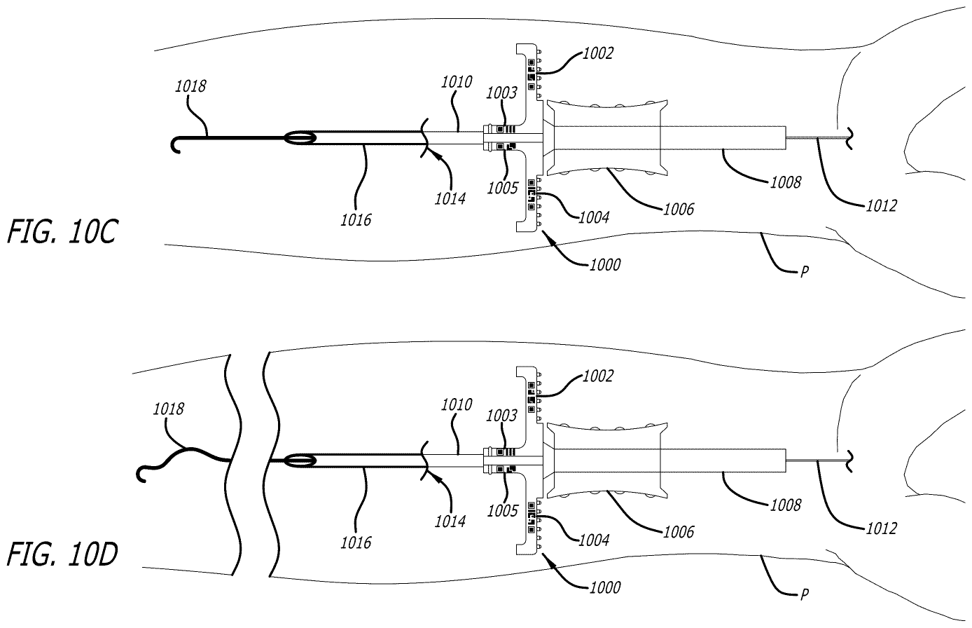

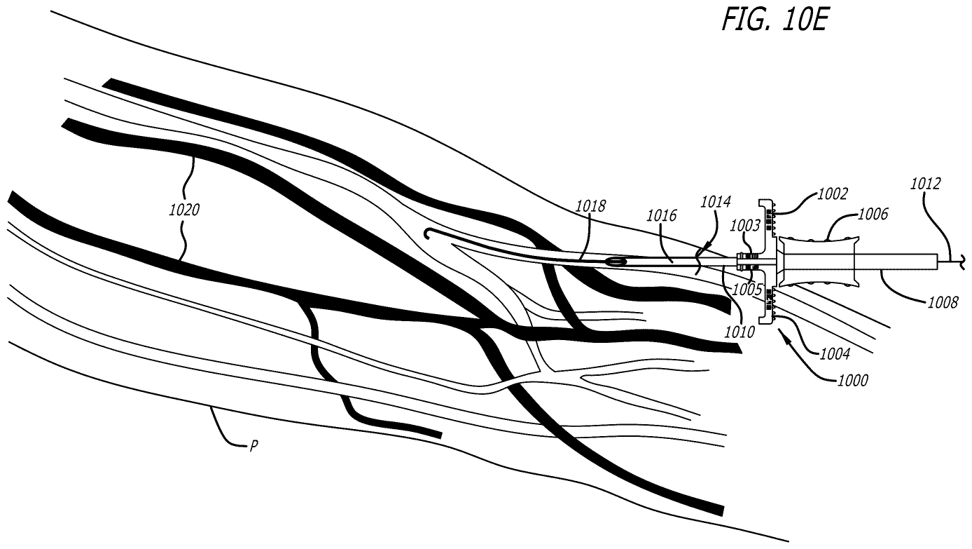

The XR headset 900 relates movements or movement data of a medical instrument to the anchor position of introducer 900 and generates a virtual representation of the medical instrument (block 980). For example, the medical instrument may be a catheter, stylet, or guidewire that comprises an optical fiber disposed therein, e.g., integrated into a wall or casing of the medical instrument, that The optical fiber includes one or more of core fibers, each of the one or more core fibers including a plurality of sensors distributed along a longitudinal length of a corresponding core fiber and each sensor of the plurality of sensors being configured to (i) reflect a light signal of a different spectral width based on received incident light, and (ii) change a characteristic of the reflected light signal based on strain experienced by the optical fiber. The movement and shape of the medical instrument may be determined may be on the changes in characteristics of the reflected light signal as discussed above, which are relayed to the XR logic 913. In particular, movement and shape of the medical instrument may be tracked as the medical instrument propagates through a patient vasculature. The XR logic 913 then generates a virtual representation of the medical instrument relative to the anchor position of the introducer 900, an example of which is illustrated in FIGS. 10C-10E.

FIGS. 10A-10E illustrate a varying views of a portion of a patient arm during a vascular access procedure. FIG. 10A illustrates a real-world view of the vascular access procedure and FIG. 10B provides the real-world view of FIG. 10A along with a conceptual line drawing of a portion of a needle and a medical instrument within the patient body. FIGS. 10A-10B illustrate an introducer 1000 placed adjacent an insertion site 1014 of patient P. The introducer 1000 is shown to include markings 1002, 1003, 1004, 1005 that are configured to be identified by an XR headset. The introducer 1000 is coupled to a finger grip 1006 and a hub 1008. A needle 1010 extends through a channel of the body of the introducer 1000 and a medical instrument 1012, such as a guidewire, is shown to be disposed within a lumen of each of the hub 1008, the finger grip 1006, and the needle 1010. FIG. 10B illustrates conceptual line drawings of a portion 1010′ of the needle 1010 and a portion 1012′ of the medical instrument 1012 that is within the body of the patient P.

FIGS. 10C-10D illustrate an XR view of the portion of the patient arm during the vascular access procedure as seen through a XR headset, such as the XR headset 900, that includes a virtual representation of a portion of the needle and the medical instrument. FIG. 10C illustrates a first state of the vascular access procedure and FIG. 10D illustrates a second state of the vascular access procedure, i.e., after propagation of the medical instrument further into the patient vasculature as compared to the first state. Finally, FIG. 10E illustrates an XR view of the portion of the patient arm during the vascular access procedure as seen through a XR headset, such as the XR headset 900. As an additional component to the displays of FIGS. 10C-10D in which a virtual representation of a portion of the needle and medical instrument are shown, FIG. 10E provides a virtual representation of the vasculature (e.g., veins and arteries) of the patient and the medical instrument propagating through the vasculature.

Referring to FIG. 10C in further detail, a third illustration of the introducer of FIG. 10A guiding the needle into the patient vasculature and the medical instrument threaded through the needle including a virtual representation of portions of the needle and the medical instrument is shown in accordance with some embodiments. FIG. 10C provides an illustration of an XR view of the vascular access procedure for which the real-world view (non-XR) is shown in FIG. 10A. Additionally, FIG. 10C provides one example of how the XR logic 913 operates to provide a medical profession (or other user) with an XR view of components of the vascular access procedure that are within the body of the patient P. While FIG. 10B provides a conceptual drawings that illustrate a portion 1010′ of the needle 1010 and a portion 1012′ of the medical instrument 1012 that is within the body of the patient P, FIG. 10C provides an example illustration of the conceptual drawings of FIG. 10B generated and displayed as corresponding virtual representations viewed through an XR headset, such as the XR headset 900.

In order to generate the virtual representation 1016 of the portion of the needle 1010 that is within the body of the patient P and the virtual representation 1018 of the portion of the medical instrument 1012 that is within the body of the patient P, the XR logic 913 perform the operations discussed with respect to FIG. 9B including capturing image data of the medical procedure environment, analyzing the image data to detect objects within the medical procedure environment, identifying the introducer 1000 at least due to the markings 1002, 1003, 1004, 1005 present on the introducer 1000, anchoring the position of the introducer 1000 in 3D space, relating movements or movement data of the medical instrument 1012 to the anchor position of introducer 1000 and generating the virtual representations 1016, 1018.

As discussed in detail above, the medical instrument 1012 includes an optical fiber cable integrated into or coupled therewith that is configured to receive light signals, e.g., from a console device, and receive reflected light signals received from sensors disposed along a length of optical fiber cable. The shape sensing logic 194 is configured to determine a shape of the medical instrument 1012 based on the reflected light signals received from the fiber optical cable. The shape data may correspond to location data within a 3D space such as coordinate (x, y, z) data points in the medical procedure environment in which the patient P is located. The XR logic 913 obtains the shape data from the console, e.g., the console 110, and generates the virtual representation 1018 of the medical instrument 1012. Additionally, the XR logic 913 may generate the virtual representation 1016 of the needle 1010 based on the shape data of the medical instrument 1012, the angle at which the needle 1010 is inserted into the patient P, and/or knowledge of the length of the needle 1010.

As noted above, FIG. 10D provides an illustration of the XR view similar to that shown in FIG. 10C. While FIG. 10C provides an illustration of the vascular access procedure at a first state in a conceptual XR view, FIG. 10D provides an illustration of the vascular access procedure at a second state in a conceptual XR view, i.e., at a subsequent time to the first state of FIG. 10 following propagation of the medical instrument further into the patient vasculature.

Referring to FIG. 10E in further detail, a fifth illustration of the introducer of FIG. 10A guiding the needle into the patient vasculature and the medical instrument threaded through the needle including a virtual representation of portions of the needle, the medical instrument, and the patient vasculature is shown in accordance with some embodiments. FIG. 10E provides an additional feature present in certain embodiments that includes generation of a virtual representation 1020 of a human vasculature (e.g., veins and arteries) that is displayed by an XR headset. In some embodiments, the virtual representation 1020 may be generated based on known, historical data of general human vasculature/anatomy with virtual representations based thereon being conceptual and generalized. In other embodiments, the virtual representation 1020 may be generated based on known, historical data of general human vasculature/anatomy in addition to ultrasound data, e.g., Doppler ultrasound data, that provides vasculature/anatomy details specific to the patient P. In yet other embodiments, the virtual representation 1020 may be generated based on known, historical data of general human vasculature/anatomy in addition to ultrasound data and/or blood flow data obtained through analysis of the reflected light signals. For example, analysis of the fluctuation or flutter of the distal tip of the medical instrument may be indicative of whether the medical instrument is traveling in a vein or artery. The fluctuation of the distal tip of a medical instrument is greater as the medical instrument moves in the direction of blood flow as opposed to moving against blood flow. Thus, as the medical instrument travels toward the heart within the vasculature of the patient P, the shape sensing logic 193, which may include specific instruments or sub-logic dedicated to fluctuation detection and analysis, may determine whether the medical instrument is traveling with or against the direction of blood flow. In some examples, the determination may be performed by machine learning models that have been trained on historical fluctuation and blood flow data.

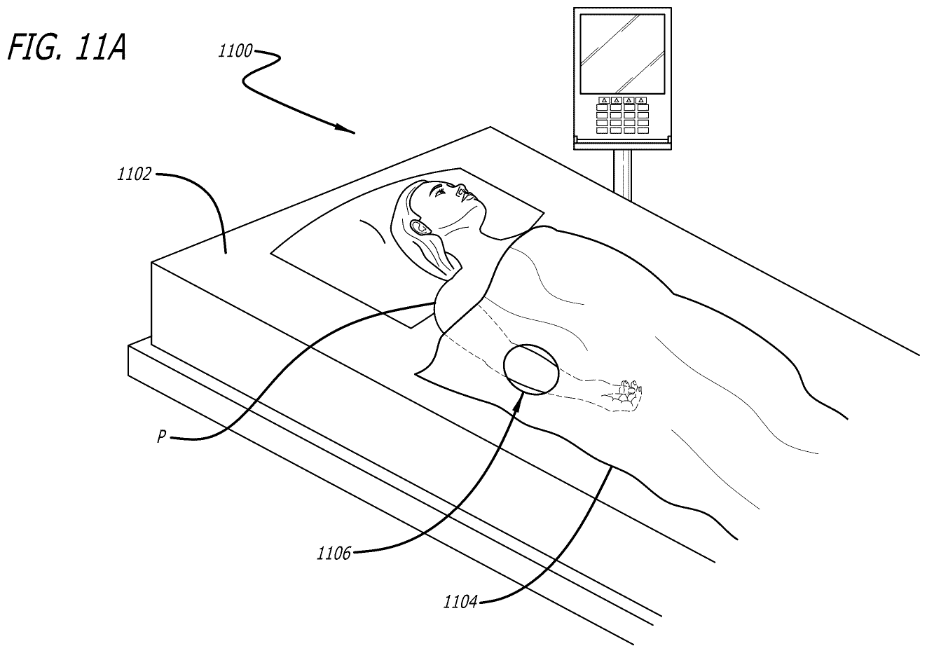

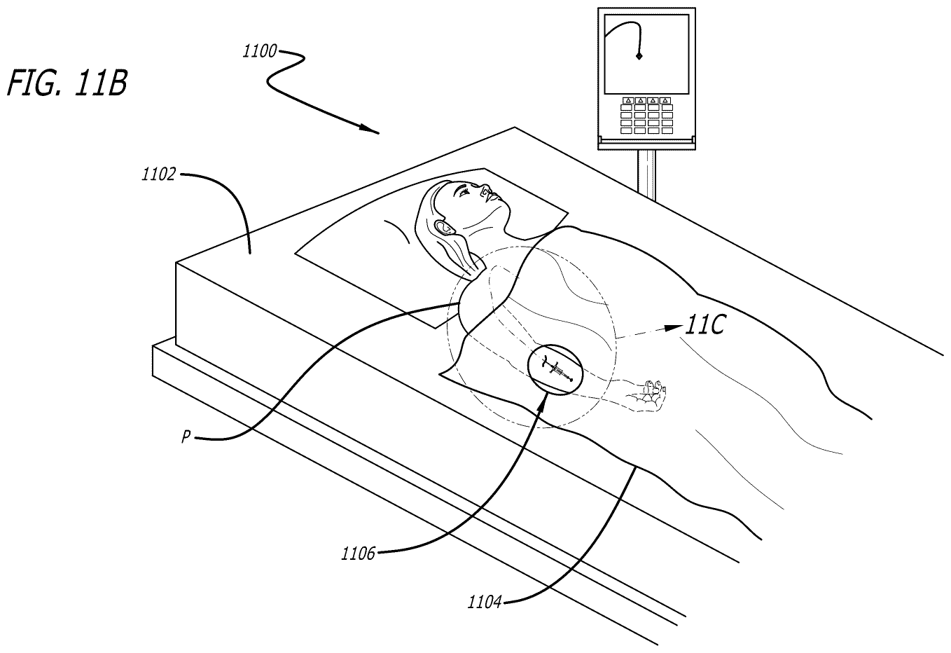

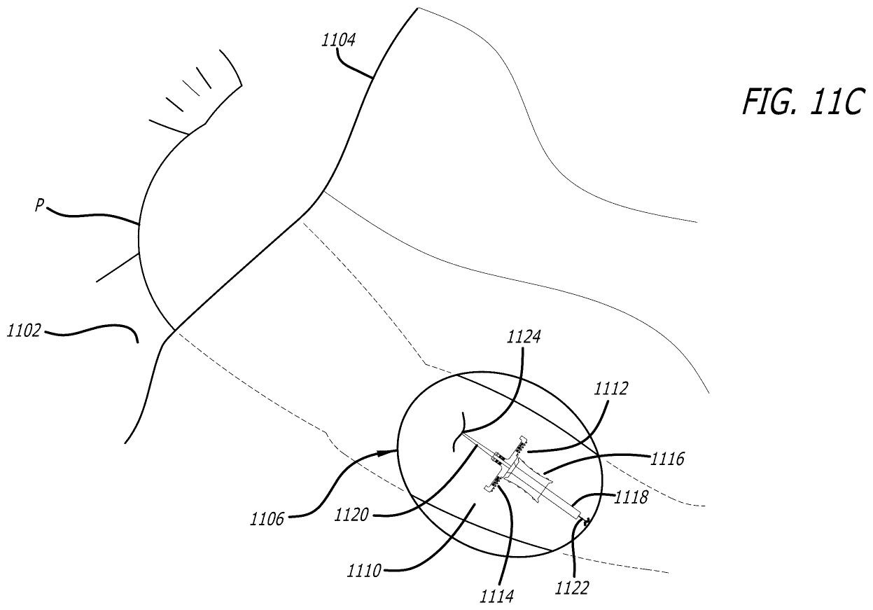

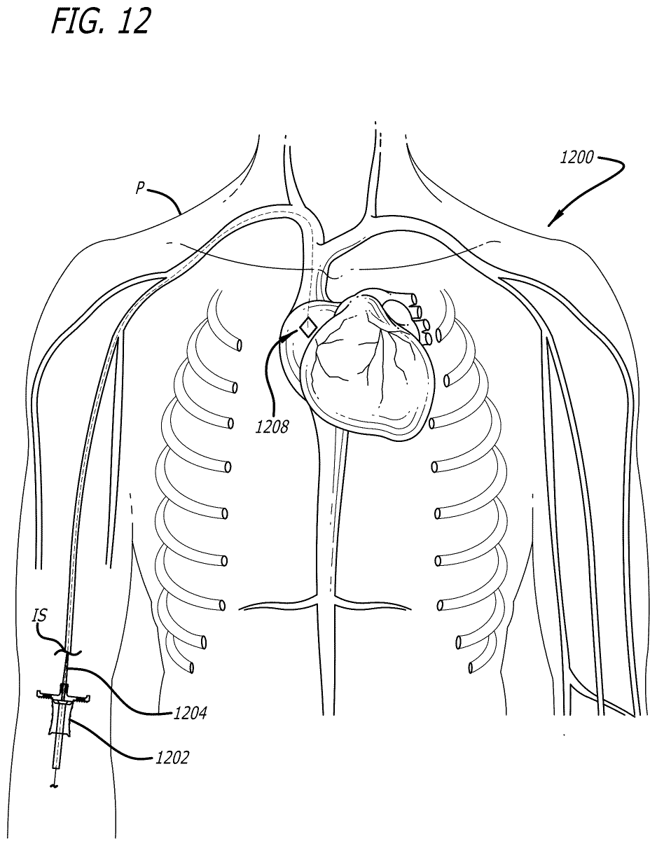

Referring now to FIG. 11A, a medical environment including a patient under a drape undergoing a vascular access procedure is shown in accordance with some embodiments. FIG. 11A provides an example illustration of a real-world view of a patient P laying on a medical bed 1102 under a drape 1104 undergoing a vascular access procedure at the procedure site 1006 within the medical environment 1100.

Referring to FIG. 11B, a view of the medical environment of FIG. 11A through an XR headset that includes a virtual representation of the medical instrument propagating through the patient vasculature is shown in accordance with some embodiments. FIG. 11B illustrates an XR view of the patient P during the vascular access procedure and in the same environment 1100 of FIG. 11A as seen through a XR headset, such as the XR headset 900, that includes virtual representations 1120′ and 1122′ of the portions of a needle 1120 and a medical instrument 1122, e.g., a guidewire having an optical fiber disposed along a length of thereof. References numerals are displayed in FIG. 11C, which provides a detailed view of the vascular access area 1006.

FIG. 11B provides an illustration of the result of performing tracking of the medical instrument through an overlay of a virtual representation of the medical instrument propagating through the vasculature of the patient P. As discussed above, the tracking of the medical instrument may be based on reflected light signals received from sensors disposed along a length of a fiber optic integrated into or coupled with the medical instrument 1122. Thus, it should be clear to one of ordinary skill in the art that FIG. 11B illustrates a technological improvement over the current state of tracking medical instruments within the patient body as discussed and a practical application that is inexplicably tied to a computing environment, namely the computer vision techniques and XR visualization is not possible outside of a computing environment.

Referring to FIG. 11C, a detailed view of a portion of FIG. 11B is shown in accordance with some embodiments. FIG. 11C provides a close-up view of a portion of FIG. 11B focusing on the medical procedure area 1106, the virtual representation 1120′ of the needle 1120, and the virtual representation 1122′ of the medical instrument 1122. Similar to other figures included herein, FIG. 11C illustrates a vascular access procedure at an insertion site 1124 that includes a needle 1120 coupled to an introducer 1110 that includes markings 1112, 1114, which are similar to the markings discussed above. The introducer 1110 is further coupled to a finger grip 1116 and a hub 1118, where each of the hub 1118, the finger grip 1116, and introducer 1110, and the needle 1120 have a medical instrument 1122 threaded through a lumen or cavity. The medical instrument 1122 continues through the needle 1120 and propagates through the vasculature of the patient P. The XR view as seen through an XR headset includes a virtual representation 1120′ of the portion of the needle 110 that is disposed within the body of the patient P and the virtual representation 1122′ of the portion of the medical instrument 1122 that is within the body of the patient P. Further, the virtual representation 1122′ changes in real-time as the medical instrument 1120 continues propagation through the body of the patient P (or as close to real-time as possible following processing of reflected signal data by the console and generation of the display by the XR headset).

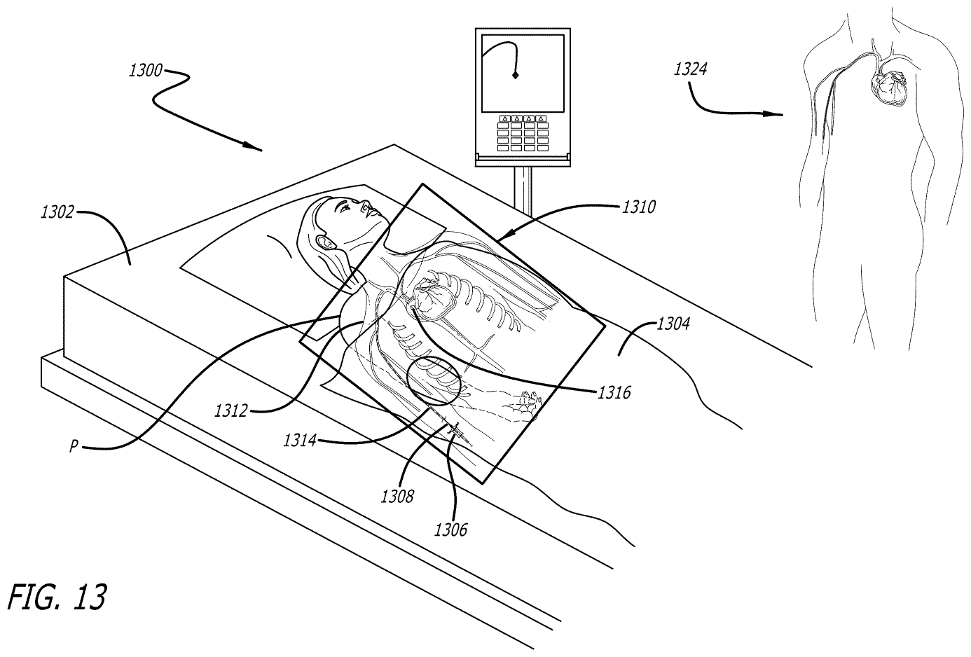

Referring now to FIG. 12, a conceptual virtual reality (VR) view of a patient vasculature and a virtual representation of a medical instrument propagating through the patient vasculature during a vascular access procedure is shown in accordance with some embodiments. FIG. 12 illustrates the conceptual VR view of a virtual anatomy (including a vasculature) and virtual representations of a medical instrument (e.g., a catheter) 1204 propagating through the vasculature 1200 placed on the virtual anatomy. The VR view of FIG. 12 is a conceptual view while FIG. 13 illustrates a practical application of the conceptual VR view of FIG. 12 as an overlay of a patient P during a vascular access procedure. In both instances, the virtual representation of the medical instrument 1204 may represent actual movement and a shape of a physical medical instrument as it propagates through the actual vasculature of an actual patient based on shape sensing procedures utilizing an optical fiber cable integrated into or coupled with the medical instrument as discussed above.

FIG. 12 illustrates an introducer 1202, similar to the introducers 800, 1000, and 1110 discussed above, the medical instrument 1204 (e.g., a catheter) entering the virtual representation of a patient body at an insertion site (IS). Additionally, the medical instrument 1204 is shown being tracked through the virtual anatomy and vasculature 1200 as it reaches a target location, which is highlighted by the target location indicator 1208. In some examples, the reflected light signal data obtained generated by an optical fiber cable integrated in or coupled with the medical instrument 1204 may be utilized to determine the location of the medical instrument 1204, including a distal tip thereof. In other implementations, various additional modalities may be utilized to confirm the location of the distal tip. An example of such modalities include electrocardiogram (ECG) technology with the distal tip of the medical instrument acting as an intravascular ECG electrode in conjunction with electrodes placed externally on a patient body (e.g., in a Lead II configuration on the right arm, left leg, and ground). An additional example include magnetic-based confirmation using a tip-location sensor (“TLS”) configured for placement on a chest of the patient and is configured with a magnetic sensor (e.g., a magnetometer array). In such an example, the medical instrument 1204 is configured with or coupled to a magnetic element, typically at the distal tip, such that the TLS detects the static magnetic field generated by the magnetic element of the distal tip of the medical instrument 1204. Such a TLS may be similar to that of TLS 50 of catheter-placement system 10 described in WO 2014/062728, which publication is incorporated by reference in its entirety into this application.

Referring now to FIG. 13, a view of the medical environment of FIG. 11A through an XR headset that includes a virtual reality (VR) view 1300 of a patient vasculature and a virtual representation of a medical instrument propagating through the patient vasculature during a vascular access procedure overlaid on the patient undergoing the vascular access procedure is shown in accordance with some embodiments. As noted above, FIG. 13 illustrates a practical application of the conceptual VR view of FIG. 12 as an overlay of a patient P during a vascular access procedure. Similar to the illustration and discussion of FIGS. 11B-11C in which a virtual representation of a portion of a medical instrument is provided as an overlay on top of a drape placed over a patient P, FIG. 13 expands on the VR display by providing an overlay 1310 of the virtual anatomy/vasculature 1312 (similar to the vasculature 1200 of FIG. 12) on a drape 1304 that is placed on a patient P who is on a medical bed 1302. As seen through an XR headset, such as the XR headset 900 of FIG. 9A, the medical instrument 1308 may be tracked according to reflected light signals obtained from an optical fiber cable integrated into or coupled to the medical instrument 1308 and a virtual representation 1314 of the portion of the medical instrument 1308 disposed in the vasculature of the patient P is generated based on shape data obtained or determined through the tracking. In some embodiments, a target location indicator 1306 may be shown, the location of which may be provided as input to the console.

In displaying the virtual anatomy/vasculature 1312 and the virtual representations (1314, 1320) of the medical instrument 1308 via the display of an XR headset, the XR logic 913 may be configured to anchor the objects of the virtual anatomy/vasculature 1312 and the virtual representation 1314 of the medical instrument 1308 to the environment or the patient P, which is characteristic of mixed reality. With respect to a medical instrument 1308 such as a guidewire or a catheter (e.g., PICC), the objects of virtual anatomy displayed in the XR view 1300 can be restricted to the circulatory system of the patient P using the Doppler imaging mode of an ultrasound probe (not shown). The XR logic 913 can be configured to track and display advancement of the medical instrument 1308 to a target location all the way up a superior vena cava (“SVC”) of the patient P proximate at least a sinoatrial node in a right atrium of a heart of the patient, which includes displaying placement of a representation of a medical instrument 1308 in an object of virtual anatomy such as a virtual SVC. While advancing the medical instrument 1308 to the target location in the patient P, a distal-end portion of the representation of the medical instrument 1308 can be configured to indicate proximity of the medical instrument 1308 to the target location in the patient by way of a visual indicator 1316 (e.g., color gradient, flashing speed, glowing intensity, etc.) of the representation of the medical instrument 1308.

As yet another optional feature of the VR view 1300, the XR headset may generate and display a holographic (virtual) representation 1324 of the body of the patient P adjacent to the medical bed 1302. In some embodiments, the holographic representation 1324 may be display less of a virtual anatomy of the patient P while also displaying a second virtual representation of the medical instrument 1308. In other embodiments, the holographic representation 1324 may duplicate the content displayed in the overlay 1310. In some instances, the holographic representation 1324 may be anchored to a location within the medical procedure environment (room) and enable a medical professional to walk around the holographic representation 1324.

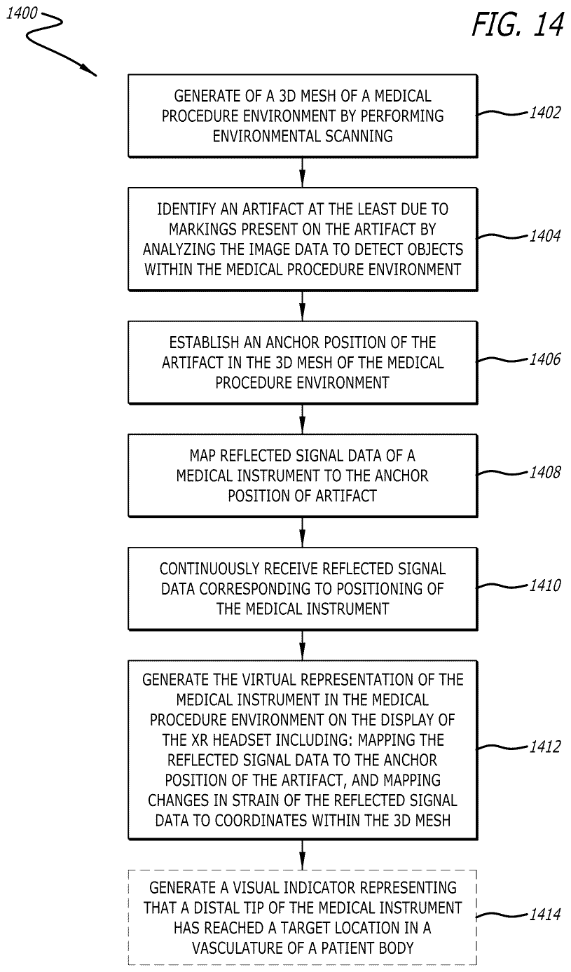

Referring now to FIG. 14, a flowchart illustrating operations performed in identifying an introducer within a 3D space as an anchor position and generating an EX display of a medical instrument originating from the anchor position is shown in accordance with some embodiments. The process 1400 can be implemented, for example, by a computing device, e.g., an XR headset 900, that comprises a processor and a non-transitory computer-readable medium. The non-transitory computer readable medium may store instructions that, when executed by the processor, can cause the processor to perform the operations of the illustrated process 1400.

Each block illustrated in FIG. 14 represents an operation in the process 1400 performed by, for example, the XR headset 900 of FIG. 9A. It should be understood that not every operation illustrated in FIG. 14 is required. In fact, certain operations may be optional to complete aspects of the process 1400. The process 1400 begins with generating a 3D mesh of a medical procedure environment by performing environmental scanning (block 1402). Generation of the 3D mesh may include operations of captures images (or video) using cameras, depth sensors, or light detection and ranging (LIDAR) to scan a real-world environment, i.e., the room in which the vascular access procedure is taking place, also referred to herein as the “medical procedure environment.” From this data, the logic of the XR headset generates a 3D mesh comprising a 3D model of surfaces, edges, and objects in real time.

An introducer present within the medical procedure environment is identified at the least due to markings present on the introducer by analyzing the image data to detect objects within the medical procedure environment (block 1404). Frames of the image data may be scanned in order to detect groups of pixels that correspond to known shapes or patterns through object recognition processing and detects objects using bounding boxes or segmentation masks, which may then be classified through machine learning techniques. In some instances, the introducer may include predefined markings that identify the introducer as well as the orientation of the introducer, which simplifies the object detection process and reduces the processing needed to identify the introducer and its orientation through other processing means.

The position of the introducer is then anchored in the 3D mesh of the medical procedure environment (block 1406). For example, an anchor position in the 3D mesh such as a fixed reference point (x, y, z coordinate) for the introducer may be recorded and a bounding box (or bounding volume) may be established around the introducer. The bounding box defines the size, position, and orientation of the introducer.

Reflected signal data of a medical instrument is received and mapped to the anchor position of introducer (block 1408). As discussed above, a medical instrument may include an optical fiber cable that includes a plurality of optical core fibers that each provide reflected light signals to a console. The reflected light signals are analyzed and converted into strain data that indicates a shape of each optical core fiber, which together may comprise data representative of a 3D shape of the medical instrument. The 3D shape of the medical instrument may then be fixed to the anchor position of the introducer. Specifically, the anchor position of the introducer (the x, y, z coordinate reference point) may act as a proximal starting point for the generation of a virtual representation of the medical instrument as discussed below.

Continuously receive reflected signal data corresponding to positioning of the medical instrument (block 1410). In response to continuously receiving the reflected signal data, a virtual representation of the medical instrument in the medical procedure environment is continuously generated and displayed on the display of the XR headset including by mapping the reflected signal data to the anchor position of the artifact, and mapping changes in strain of the reflected signal data to coordinates within the 3D mesh (block 1412). As a result, a virtual representation of the medical instrument is generated in real-time as the medical instrument propagates through a vasculature of a patient body. Further, as the virtual representation of the medical instrument is fixed to the anchor position of the introducer, the virtual representation is positioned as an overlay on the patient body (or on a drape covering the patient body in some instances) that provides a medical professional an indication of where the medical instrument is in patient body.

As an optional additional operation, a visual indicator representing that a distal tip of the medical instrument has reached a target location in a vasculature of a patient body may be generated (block 1414). For example, the visual indicator may be a shape having a specific color, a pattern color gradient, a flashing brightness, a glowing intensity, etc., and may be positioned at a distal tip of the virtual representation of the medical instrument. In some implementations, the visual indicator is generated following receipt of data obtained through a second modality, such as electrocardiogram (ECG) signal data, that indicates a distal tip of the medical instrument is positioned at a target location such as in the lower one-third portion of the SVC of the patient for example.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A computerized method for displaying a virtual representation of a medical instrument on a display of an extended reality (XR) headset, the computerized method comprising:
generating a three-dimensional (3D) mesh of a medical procedure environment by performing environmental scanning;
identifying an artifact within the medical procedure environment;
establishing an anchor position of the artifact within the 3D mesh;
continuously receiving reflected signal data corresponding to positioning of the medical instrument; and
generating the virtual representation of the medical instrument in the medical procedure environment on the display of the XR headset including:
mapping the reflected signal data to the anchor position of the artifact, and
mapping changes in strain of the reflected signal data to coordinates within the 3D mesh.

2. The computerized method of claim 1, wherein the artifact is an introducer disposed adjacent to an insertion site for a vascular access procedure.

3. The computerized method of claim 2, wherein the artifact includes markings, and wherein identifying the artifact includes detecting the markings using computer vision techniques.

4. The computerized method of claim 1, wherein generating the 3D mesh includes performing the environmental scanning by capturing image data with one or more cameras of the XR headset.

5. The computerized method of claim 1, wherein the medical instrument includes a multi-core optical fiber including a plurality of core fibers that generate the reflected signal data.

6. The computerized method of claim 5, wherein each of the plurality of core fibers includes a plurality of sensors distributed along a longitudinal length of a corresponding core fiber and each sensor of the plurality of sensors being configured to (i) reflect a light signal of a different spectral width based on received incident light, and (ii) change a characteristic of the reflected light signal based on strain experienced by the core fiber.

7. The computerized method of claim 1, wherein the virtual representation of the medical instrument represents shape and placement of a portion of the medical instrument that is inside a vasculature of a patient during a vascular access procedure.

8. An extended reality (XR) apparatus comprising:
a housing coupled to a display, a head strap, and one or more cameras;
one or more processors housed within the housing; and
non-transitory computer-readable medium having stored thereon logic that, when executed by one or more processors, causes operations for displaying a virtual representation of a medical instrument on the display, the operations including:
generating a three-dimensional (3D) mesh of a medical procedure environment by performing environmental scanning,
identifying an artifact within the medical procedure environment,
establishing an anchor position of the artifact within the 3D mesh,
continuously receiving reflected signal data corresponding to positioning of the medical instrument, and
generating the virtual representation of the medical instrument in the medical procedure environment on the display of the XR headset including:
mapping the reflected signal data to the anchor position of the artifact, and
mapping changes in strain of the reflected signal data to coordinates within the 3D mesh.

9. The XR apparatus of claim 8, wherein the artifact is an introducer disposed adjacent to an insertion site for a vascular access procedure.

10. The XR apparatus of claim 9, wherein the artifact includes markings, and wherein identifying the artifact includes detecting the markings using computer vision techniques.

11. The XR apparatus of claim 8, wherein generating the 3D mesh includes performing the environmental scanning by capturing image data with one or more cameras of the XR headset.

12. The XR apparatus of claim 8, wherein the medical instrument includes a multi-core optical fiber including a plurality of core fibers that generate the reflected signal data.

13. The XR apparatus of claim 12, wherein each of the plurality of core fibers includes a plurality of sensors distributed along a longitudinal length of a corresponding core fiber and each sensor of the plurality of sensors being configured to (i) reflect a light signal of a different spectral width based on received incident light, and (ii) change a characteristic of the reflected light signal based on strain experienced by the core fiber.

14. The XR apparatus of claim 8, wherein the virtual representation of the medical instrument represents shape and placement of a portion of the medical instrument that is inside a vasculature of a patient during a vascular access procedure.

15. A non-transitory computer-readable medium having stored thereon logic that, when executed by one or more processors, causes operations for displaying a virtual representation of a medical instrument on a display of an extended reality (XR) headset, the operations including:
generating a three-dimensional (3D) mesh of a medical procedure environment by performing environmental scanning;
identifying an artifact within the medical procedure environment;
establishing an anchor position of the artifact within the 3D mesh;
continuously receiving reflected signal data corresponding to positioning of the medical instrument; and
generating the virtual representation of the medical instrument in the medical procedure environment on the display of the XR headset including:
mapping the reflected signal data to the anchor position of the artifact, and
mapping changes in strain of the reflected signal data to coordinates within the 3D mesh.

16. The non-transitory computer-readable medium of claim 15, wherein the artifact is an introducer disposed adjacent to an insertion site for a vascular access procedure, wherein the introducer includes markings, and wherein identifying the artifact includes detecting the markings using computer vision techniques.

17. The non-transitory computer-readable medium of claim 15, wherein generating the 3D mesh includes performing the environmental scanning by capturing image data with one or more cameras of the XR headset.

18. The non-transitory computer-readable medium of claim 15, wherein the medical instrument includes a multi-core optical fiber including a plurality of core fibers that generate the reflected signal data.

19. The non-transitory computer-readable medium of claim 18, wherein each of the plurality of core fibers includes a plurality of sensors distributed along a longitudinal length of a corresponding core fiber and each sensor of the plurality of sensors being configured to (i) reflect a light signal of a different spectral width based on received incident light, and (ii) change a characteristic of the reflected light signal based on strain experienced by the core fiber.

20. The non-transitory computer-readable medium of claim 15, wherein the virtual representation of the medical instrument represents shape and placement of a portion of the medical instrument that is inside a vasculature of a patient during a vascular access procedure.
